(19) United States
(12) Patent Application Publication
(10) Pub. No.: US 2024/0408317 A1
(43) Pub. Date: Dec. 12, 2024

(54) A sub-assembly of a medicament delivery device cassette unit and a method of operating a medicament delivery device cassette unit comprising the sub-assembly

(71) Applicant: SHL Medical AG, Zug (CH)

(72) Inventor: Ming-Ting Yin, Taoyuan City (TW)

(21) Appl. No.: 18/697,921

(22) PCT Filed: Sep. 30, 2022
(86) PCT No.: PCT/EP2022/077254

§ 371 Date: Apr. 2, 2024

(30) Foreign Application Priority Data
Oct. 14, 2021 (EP) 21202568.8
Oct. 14, 2021 (EP) 21202570.4

(51) Int. Cl. A61M 5/315

(52) U.S. Cl. CPC A61M 5/31576

(57) ABSTRACT

The present disclosure provides a sub-assembly of a medicament delivery device cassette unit, the sub-assembly including a tubular body for attaching to a drive unit of a medicament delivery device. The tubular body extends along a longitudinal axis between a proximal end and a distal end. The sub-assembly further includes a tubular carrier for holding a medicament container. The tubular carrier is at least partially arranged within the tubular body, and the tubular carrier and the tubular body are coaxial. The tubular carrier comprises a distally directed surface for contacting with the drive unit of the medicament delivery device when the tubular body being attached to the drive unit of the medicament delivery devices. The tubular carrier is movable relative to the tubular body in the direction of the longitudinal axis from a distal position to a proximal position by the drive unit when the distally directed surface is in contact with the drive unit of the medicament delivery device.

### TECHNICAL FELD

The present disclosure generally relates to a sub-assembly of a medicament delivery device cassette unit and a method of operating a medicament delivery device cassette unit comprising the sub-assembly, and particularly to sub-assemblies with a component that is moved when the medicament delivery device cassette unit is attached to a medicament delivery device drive unit.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments such as a growth hormone.

Some medicament delivery devices have two parts that are connected by the patient prior to use for administration of the medicament. A medicament container, such as a cartridge or a syringe, is often inserted or pre-installed in one part. This part usually can be called a cassette or a cassette unit. The other part often comprises a driver for actuating expulsion of the medicament. Often, a medicament container that contains a medicament, such as a cartridge or a syringe, is inserted in one part of the delivery device or comes pre-installed in said part. The part that receives the medicament container that contains a medicament is often called a cassette or a cassette unit.

Medicament delivery devices designed in this way are commonly reusable. The cassette is a disposable part, and the other part with the driver usually is reusable. The driver may comprise a power unit such as a spring or a motor and thus can be re-used for some medicament deliveries.

There is room for improvement of current cassette design in the market, such as adding more automatic functions and minimisation or reduction of the cost of the cassette.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or“circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device cassette unit, the sub-assembly comprising: a tubular body for attaching to a drive unit of a medicament delivery device; wherein the tubular body extends along a longitudinal axis between a proximal end and a distal end; and a tubular carrier for holding a medicament container; wherein the tubular carrier is at least partially arranged within the tubular body; wherein the tubular carrier and the tubular body are coaxial; wherein the tubular carrier comprises a distally directed surface for contacting with the drive unit of the medicament delivery device when the tubular body being attached to the drive unit of the medicament delivery device; wherein the tubular body comprises a flexible section at the distal end of the tubular body; and wherein the flexible section comprises a hook extending in a radial direction relative to the longitudinal axis and a lock protrusion extending away from the tubular body in the radial direction relative to the longitudinal axis; wherein the tubular carrier comprises a carrier body; wherein a cut-out or recess is arranged in a wall of the carrier body; and wherein the flexible section is movable in a direction transverse to the longitudinal axis relative to the tubular body between an engaged position where the hook of the flexible section is positioned within the cut-out or recess of the tubular carrier and a released position where the hook of the flexible section is positioned out of the cut-out or recess of the tubular carrier; and wherein the flexible section of the tubular body is moved from the engaged position to the released position when the lock protrusion of the flexible section is pressed from the outside of the tubular body.

Another aspect of the invention provides a sub-assembly of a medicament delivery device cassette unit, the sub-assembly comprising: a tubular body for attaching to a drive unit of a medicament delivery device; the tubular body extends along a longitudinal axis between a proximal end and a distal end; and a tubular carrier for holding a medicament container; the tubular carrier is at least partially arranged within the tubular body; the tubular carrier and the tubular body are coaxial; wherein the tubular carrier comprises a distally directed surface for contacting with the drive unit of the medicament delivery device when the tubular body being attached to the drive unit of the medicament delivery device; and wherein the tubular carrier is movable relative to the tubular body in the direction of the longitudinal axis from a distal position to a proximal position by the drive unit when the distally directed surface is in contact with the drive unit of the medicament delivery device.

Preferably, according to another embodiment, the tubular carrier is engaged with the tubular body through a groove and ledge engagement.

Preferably, according to another embodiment, the tubular carrier comprises a ledge extending from an outer surface of a wall of the tubular carrier in a radial direction relative to the longitudinal axis; a groove is arranged on an inner wall of the tubular body; and the ledge is positioned within the groove.

Alternatively, according to another embodiment, the tubular body comprises a ledge extending from an inner surface of a wall of the tubular body in a radial direction relative to the longitudinal axis; wherein a groove is arranged on an outer surface of the tubular carrier; and wherein the ledge is positioned within the groove.

Preferably, according to another embodiment, the groove is a recess or a slot.

Preferably, according to another embodiment, the tubular body comprises a ledge extending from an inner surface of a wall of the tubular body in a radial direction relative to the longitudinal axis; the tubular carrier comprises a counter ledge arranged on an outer surface of the tubular carrier; and wherein the ledge is positioned within the counter ledge.

Preferably, according to another embodiment, the tubular carrier is configured to immovably hold the medicament container of the medicament delivery device.

Preferably, according to another embodiment, the sub-assembly comprises a delivery member assembly; the delivery member assembly comprises a delivery member and a delivery member holder; the delivery member is attached to the delivery member holder; and the delivery member holder is attached the proximal end of the tubular body.

Preferably, according to another embodiment, the tubular carrier is axially movable relative to the delivery member assembly between the distal position and the proximal position; the tubular carrier is configured to hold the medicament container in a non-fluid connection with the delivery member when the tubular carrier is in the distal position; and the tubular carrier is configured to hold the medicament container in a fluid connection with the delivery member when the tubular carrier is in the proximal position.

Preferably, according to another embodiment, the tubular body comprises a flexible section at the distal end of the tubular body; and the flexible section comprises a hook extending in a radial direction relative to the longitudinal axis and a lock protrusion extending away from the tubular body in the radial direction relative to the longitudinal axis.

Preferably, according to another embodiment, the tubular carrier comprises a carrier body; a cut-out or recess is arranged in a wall of the carrier body; and the flexible section is movable in a direction transverse to the longitudinal axis relative to the tubular body between an engaged position where the hook of the flexible section is positioned within the cut-out or recess of the tubular carrier and a released position where the hook of the flexible section is positioned out of the cut-out or recess of the tubular carrier.

Preferably, according to another embodiment, the flexible section of the tubular body is moved from the engaged position to the released position when the lock protrusion of the flexible section is pressed from the outside of the tubular body.

Preferably, according to another embodiment, the sub-assembly comprises a delivery member guard telescopically arranged in the tubular body and a biasing member extending along the longitudinal axis between a proximal end and a distal end; and the delivery member guard comprises a distally directed surface adjacent to the proximal end of the biasing member.

Preferably, according to another embodiment, the tubular carrier comprises a proximally directed surface adjacent to the distal end of the biasing member.

Alternatively, according to another embodiment, the tubular body comprises a proximally directed surface adjacent to the distal end of the biasing member.

Preferably, according to another embodiment, the tubular carrier comprises a flange extending in a direction transverse to the longitudinal axis; the distally directed surface is defined by the flange; and wherein the flange extends out of the tubular body.

Preferably, according to another embodiment, the tubular body comprises a fastener on an outer surface of the tubular body for attaching to a part of a medicament delivery device.

Preferably, according to another embodiment, the fastener is a snap-fit connector, a thread screw or a bayonet connector.

Preferably, according to another embodiment, the flange of the tubular carrier is arranged next to the fastener.

Preferably, according to another embodiment, the tubular carrier is movable relative to the tubular body in the direction of the longitudinal axis from a distal position to a proximal position by the drive unit when the distally directed surface is in contact with the drive unit of the medicament delivery device.

Preferably, according to another embodiment, the distally directed surface is in contact with the drive unit of the medicament delivery device when the tubular body is attached to the part of the medicament delivery device via the fastener.

Preferably, according to another embodiment, the flange is circumferentially offset to the bayonet connector of the tubular body relative to the longitudinal axis.

Preferably, according to another embodiment, the delivery member is a needle; and wherein the delivery member assembly comprises a flexible sheath wrapping the needle.

Preferably, according to another embodiment, the sub-assembly comprises a rotator rotatable relative to the delivery member guard around the longitudinal axis between the locked position and the unlocked position.

Preferably, according to another embodiment, the cassette unit sub-assembly comprises a biasing member arranged between a first distally directed surface of the delivery member guard and a proximally directed surface of the body.

Another aspect of the invention provides a sub-assembly of a medicament delivery device cassette unit, the sub-assembly comprising: a tubular body, a delivery member guard, and a rotator; the tubular body extends along a longitudinal axis between a proximal end and a distal end; the tubular body is tubular; the delivery member guard is telescopically arranged within the tubular body; the delivery member guard comprises a distally directed surface; the rotator is blocked from moving in either direction along the longitudinal axis by the tubular body; and the rotator comprises a proximally directed surface; and the rotator is configured to be rotated by a drive unit of the medicament delivery device when the medicament delivery device cassette unit is attached to the drive unit, so that the rotator is rotated relative to the delivery member guard from a locked position where the distally directed surface of the delivery member guard is aligned to the proximally directed surface of the rotator to a unlocked position where the distally directed surface of the delivery member guard is not aligned from the proximally directed surface of the rotator so that the delivery member guard is free to move in the distal direction relative to the rotator.

The sub-assembly can therefore provide a safety mechanism for medicament cassette unit. Especially for a medicament delivery cassette unit without a front protecting cap. The rotator will block the delivery member guard from moving in the distal direction relative to the tubular body before the cassette unit being properly attached to a drive unit of the medicament delivery device, so that an end user will not accidentally contact a medicament delivery member that is shielded by the medicament delivery member guard.

Preferably, according to another embodiment, the rotator comprises a tubular rotator body.

Preferably, according to another embodiment, the rotator comprises a ledge radially extending relative to the longitudinal axis; and the ledge is configured to engage with a part of the drive unit, so that when the drive unit rotates relative to the tubular body of the sub-assembly, the rotator is rotated together with the drive unit relative to the tubular body of the sub-assembly.

Alternatively, according to another embodiment, the rotator comprises a ledge extending from a first end to a second end in a helical direction relative to the longitudinal axis; and the ledge is configured to engage with a part of the drive unit, so that when the drive unit of the medicament delivery device axially moves relative to the rotator from a distal position where the part of the drive unit is adjacent to the first end of the ledge and a proximal position where the part of the drive unit is adjacent to the second end of the ledge, thereby the rotator is rotated.

Preferably, according to another embodiment, the sub-assembly comprises a lock member; the lock member is rotationally fixed to the tubular body; one of the rotator and the lock member comprises a protrusion radially extending relative to the longitudinal axis; the other one of the rotator and the lock member comprises a ledge adjacent to the protrusion; the ledge extending from a first end to a second end in a helical direction relative to the longitudinal axis; the lock member is axially movable relative to the rotator from a distal position where the protrusion is adjacent to the first end of the ledge and a proximal position where the protrusion is adjacent to the second end of the ledge; and wherein the lock member is configured to engage with a part of the drive unit, so that when the drive unit of the medicament delivery device axially moves relative to the tubular body, the lock member moves together with the drive unit, thereby the rotator is rotated by the engagement between the protrusion and the ledge.

Preferably, according to another embodiment, one of the rotator and the delivery member guard comprises a protrusion, and the other one of the rotator and the delivery member guard comprises a cut-out or recess; and the distally directed surface of the delivery member guard and the proximally directed surface of the rotator are defined by the protrusion and the cut-out or recess respectively.

Preferably, according to another embodiment, the cut-out or recess is arranged on the tubular body of the rotator.

Preferably, according to another embodiment, the ledge is extending from the tubular body of the rotator in the radial direction relative to the longitudinal axis.

Alternatively, according to another embodiment, a second cut-out or recess is arranged on the tubular body of the rotator; and wherein the ledge is defined by an edge of the second cut-out or recess.

Alternatively, according to another embodiment, a circumferential ledge extends in a circumferential direction relative to the longitudinal axis from the second end of the ledge.

Preferably, according to another embodiment, the first end of the ledge comprises a longitudinal part extending in the direction of the longitudinal axis.

Preferably, according to another embodiment, the sub-assembly comprises a biasing member extending along the longitudinal axis between a proximal end and a distal end; the delivery member guard comprises a distally directed surface; the lock member comprises a proximally directed surface; and the proximal end of the biasing member is adjacent to the distally directed surface of the delivery member guard; and the distal end of the biasing member is adjacent to the proximally directed of the lock member.

Preferably, according to another embodiment, the sub-assembly comprises a biasing member arranged between a first distally directed surface of the delivery member guard and a proximally directed surface of the body.

Preferably, according to another embodiment, the tubular body comprises a fastener on an outer surface of the tubular body for attaching to a part of a medicament delivery device.

Preferably, according to another embodiment, the fastener can be a thread screw, a snap-fit connector or a bayonet connector.

Preferably, according to another embodiment, the lock member comprises a lock member body and a flange extending from the lock member body; wherein the flange protrudes from the outer surface of the tubular body; and wherein the flange is arranged next to the fastener.

Preferably, according to another embodiment, the flange is circumferentially offset to the bayonet connection of the tubular body relative to the longitudinal axis.

Preferably, according to another embodiment, the tubular body comprises a flexible arm extending from the distal end of the tubular body towards the proximal end of the tubular body; and the flexible arm comprises a hook extending in the radial direction relative to the longitudinal axis, and a lock protrusion extending to the outside of the tubular body in the radial direction relative to the longitudinal axis.

Preferably, according to another embodiment, the lock member comprises a lock member body; a cut-out or recess is arranged in a wall of the lock member body; and the hook of the flexible section of the tubular body is configured to position within the cut-out or recess; and the lock protrusion of the flexible section is configured to engage with a part of a driver unit of the medicament delivery device, so that when the tubular body is attached to the drive unit of the medicament delivery device, a section of the drive unit presses the lock protrusion inwardly relative to the tubular body, thus the hook flexes out from the cut-out or recess.

Preferably, according to another embodiment, the rotator is rotatable relative to the delivery member guard around the longitudinal axis between the locked position and the unlocked position.

Preferably, according to another embodiment, the delivery member guard comprises a protrusion radially extending relative to the longitudinal axis; and the proximally directed surface of the delivery member guard is a part of the protrusion.

Preferably, according to another embodiment, the delivery member guard is movable relative to the tubular body between a distal trigger position and a proximal after-use position; and the delivery member guard comprises a distally directed surface.

Another embodiment of the invention provides a cassette unit sub-assembly for a medicament delivery device, the cassette unit sub-assembly comprising: a body extending along a longitudinal axis from a proximal end to a distal end; a delivery member guard coaxially attached to the body and being proximally biased to axially move relative to the body from a distal position to a proximal position; and a rotator attached to the body; the rotator is movable relative to the delivery member guard between a locked position and an unlocked position; and the rotator is configured to lock the delivery member guard in the distal position when the rotator is in the locked position.

Preferably, according to another embodiment, the rotator is configured to lock the delivery member guard in the proximal after-use position when the delivery member guard moves into the proximal after-use position.

Preferably, according to another embodiment, the cassette unit sub-assembly of this invention can be used with a cassette unit for a medicament delivery device. The medicament delivery device comprises the cassette unit. The cassette unit can be attached and detached to a medicament delivery device drive unit.

Preferably, according to another embodiment, the cassette unit of this invention can be used with a medicament delivery device comprising a reusable drive assembly; the reusable drive assembly comprises a tubular housing extending along the longitudinal axis from a proximal end and a distal end; and the cassette unit is releasably attached to the reusable drive assembly through a bayonet connection on an inner surface on the proximal end of the reusable drive assembly and on an outer surface on the distal end of the body of the cassette unit.

According to another embodiment, the inner surface on the proximal end of the reusable drive assembly comprises a bayonet groove; and wherein the outer surface on the distal end of the body of the cassette unit comprises a bayonet protrusion.

Preferably, according to another embodiment, the rotator comprises a track comprising a lockout section, the protrusion of the delivery member guard is configured to move into the lockout section together with the distal movement of the delivery member guard towards a proximal after-use position of the delivery member guard.

Preferably, according to another embodiment, the protrusion is arranged at a flexible section of the delivery member guard.

Preferably, according to another embodiment, the medicament delivery device is an injection device, an inhalation device, or a medical sprayer.

Preferably, according to another embodiment, the medicament delivery device cassette unit is for accommodating a medicament container.

Preferably, according to another embodiment, the drive unit comprises a driver for forcing the medicament contained in the medicament container within the cassette unit to be expelled.

Preferably, according to another embodiment, the sub-assembly comprises a distal id attachable to the distal end of the body.

Preferably, according to another embodiment, the distal lid is a part of the body of the sub-assembly.

Preferably, according to another embodiment, the distal lid comprises a id body formed in a shape following the shape of the medicament container received within the body.

Preferably, according to another embodiment, the lid body comprises a tubular section.

Preferably, according to another embodiment, the distal lid comprises a container support for supporting the medicament container received within the body; the container support comprises a proximally directed surface.

Preferably, according to another embodiment, the container support is a flexible arm.

Preferably, according to another embodiment, the flexible arm of the container support can be formed by a cut-out on the lid body opening in the direction transverse to the longitudinal axis on the id body.

Preferably, according to another embodiment, the flexible arm may extend from the id body in the distal direction.

Preferably, according to another embodiment, the proximally directed surface of the container support is formed on an inner surface of the flexible arm or formed on a distal tip of the flexible arm.

Preferably, according to another embodiment, the container support is configured to prevent an axial movement in the distal direction of the medicament container received within the body and/or be used to tolerate variations in dimensions of the received medicament container, e.g. certain length and/or width of the received medicament container, regarding the engineering tolerance.

Preferably, according to another embodiment, the medicament delivery device using the subject sub-assembly of this invention can be operated by a method comprising the step of connecting the cassette unit to the drive unit.

Another aspect of the invention provides a method of operating a medicament delivery device comprising a medicament delivery device cassette unit and a drive unit, wherein the medicament delivery device cassette unit comprises a body and a carrier; the method comprising the following sequence of steps of: providing a medicament delivery device cassette unit; providing a drive unit; and using the drive unit to move the carrier of the medicament delivery device cassette unit relative to the body of the medicament delivery device cassette unit along a longitudinal axis until the medicament delivery device cassette unit attaches to the drive unit.

Preferably, according to another embodiment, the step using the drive unit to move the carrier of the medicament delivery device cassette unit relative to the body of the medicament delivery device cassette unit along the longitudinal axis until the medicament delivery device cassette unit attaches to the drive unit comprises the steps of: moving the medicament delivery device cassette unit relative to the drive unit in the direction of the longitudinal axis; and rotating the medicament delivery device cassette unit relative to the drive unit around the longitudinal axis.

Preferably, according to another embodiment, the step of using the drive unit to move the carrier comprises the steps of: connecting the carrier to a part of the drive unit; and moving the carrier during rotating the medicament delivery device cassette unit relative to the drive unit around the longitudinal axis.

Preferably, according to another embodiment, the step of using the drive unit to move the carrier of the medicament delivery device cassette unit relative to the body of the medicament delivery device cassette unit along the longitudinal axis until the medicament delivery device cassette unit attaches to the drive unit comprising the step of using the movement of the carrier relative to the body to establish a fluid connection between a delivery member to the medicament container while the medicament delivery device cassette unit attaches to the drive unit.

Another aspect of the invention provides a method of operating a medicament delivery device comprising a medicament delivery device cassette unit and a drive unit; wherein the delivery device cassette unit comprises a tubular body, a delivery member guard, and a rotator; the method comprising the following sequence of the steps of providing the medicament delivery device cassette unit; providing the drive unit; and using the drive unit to rotate the rotator of the medicament delivery device cassette unit relative to the tubular body of the medicament delivery device cassette unit whilst attaching the medicament delivery device cassette unit to the drive unit.

Preferably, according to another embodiment, the step using the drive unit to rotate the rotator of the medicament delivery device cassette unit relative to the tubular body of the medicament delivery device cassette unit whilst attaching the medicament delivery device cassette unit to the drive unit comprises the following sequence of the steps of: moving the medicament delivery device cassette unit relative to the drive unit along a longitudinal axis; and rotating the medicament delivery device cassette unit relative to the drive unit around the longitudinal axis.

Preferably, according to another embodiment, the step of using the drive unit to rotate the rotator of the medicament delivery device cassette unit relative to the tubular body of the medicament delivery device cassette unit whilst attaching the medicament delivery device cassette unit to the drive unit comprises the steps of:
establishing a connection between the rotator and a part of the drive unit; and
rotating the medicament delivery device cassette unit relative to the drive unit around the longitudinal axis, whereby the rotator is rotated by the drive unit through the connection between the rotator and the drive unit.

Preferably, according to another embodiment, the step of establishing a connection between the rotator and a part of the drive unit comprises the steps of: indirectly connecting the rotator to the part of the drive unit via a lock member of the medicament delivery device cassette unit; and rotating the medicament delivery device cassette unit relative to the drive unit around the longitudinal axis, thereby the lock member is axially moved in the proximal direction of the rotator by the drive unit, and thereby the rotator is rotated by the lock member.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device cassette unit comprising a sub-assembly of the invention.

FIG. 2 schematically shows an exploded view of the sub-assembly of FIG. 1.

FIG. 3 schematically shows a perspective view of a medicament delivery device cassette unit comprising the sub-assembly of the invention in another example.

FIG. 4 schematically shows an exploded view of the sub-assembly of FIG. 3.

FIGS. 5-8 schematically show perspective views of a tubular body of the sub-assembly of the invention in different examples.

FIGS. 7A-7B schematically show perspective views of a delivery member assembly of the sub-assembly of the invention.

FIGS. 8-9 schematically show perspective views of a tubular carrier of the sub-assembly of the invention in different examples.

FIG. 10 schematically shows a perspective view of the tubular carrier of FIG. 9 and an outer shell of the tubular body of FIGS. 5-8.

FIG. 11A schematically shows a perspective view of a delivery member guard, a biasing member, and the tubular carrier of the sub-assembly of the invention.

FIG. 11B schematically shows a cross-section view of the delivery member guard, the biasing member, and the tubular body of the sub-assembly of the invention in another example.

FIG. 11C schematically shows a cross-section view of the arrangement of FIG. 11A with the delivery member assembly of FIGS. 7A-7B.

FIGS. 12A-13B schematically show side views and cross-section views of the sub-assembly of the invention before a cassette unit is attached to a drive unit and after the cassette unit is attached to the drive unit.

FIGS. 14A-15B schematically show perspective views of a rotator of the sub-assembly of the invention in different examples.

FIG. 16 schematically show a perspective view of the delivery member guard of FIG. 11B.

FIGS. 17A-17B schematically show perspective views of a connection between the rotator of FIGS. 14A-15B and the delivery member guard of FIG. 16.

FIGS. 18A-18B schematically show perspective views of a lock member of the sub-assembly of the invention in different examples.

FIGS. 19A-19B schematically show perspective views of the lock member of FIGS. 18A-18B and the rotator of FIGS. 14A-15B. The lock member in different examples as shown in FIGS. 18A-18B can be used with the rotator as shown in either FIGS. 14A-14B or FIGS. 15A-15B with the connection as shown in FIGS. 19A-19B.

FIGS. 20A-20F schematically show a sequence of interaction between the lock member and the rotator. The lock member as shown in FIGS. 20A-20F can be the exemplified lock member as shown in either FIG. 18A or FIG. 18B. The rotator as shown in FIGS. 20A-20F can be the exemplified rotator as shown in either FIGS. 14A-14B or FIGS. 15A-15B.

FIG. 21 schematically shows a perspective view of a connection between the rotator of FIGS. 14A-15B and the tubular body of FIGS. 5-8.

FIG. 22 schematically shows a perspective view of a drive unit and the cassette unit with the sub-assembly of the invention.

FIGS. 23-24 schematically show perspective views of the tubular body, the tubular carrier and the delivery member guard in different examples.

### DETAILED DESCRIPTION

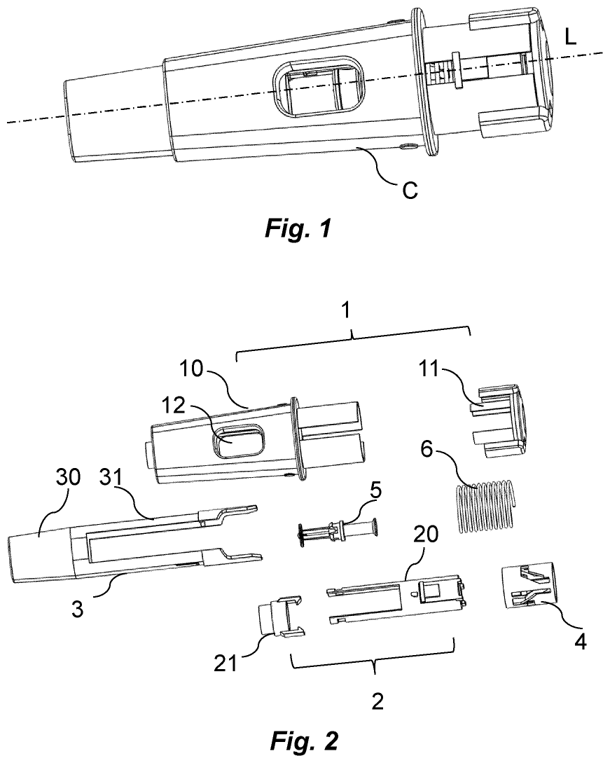

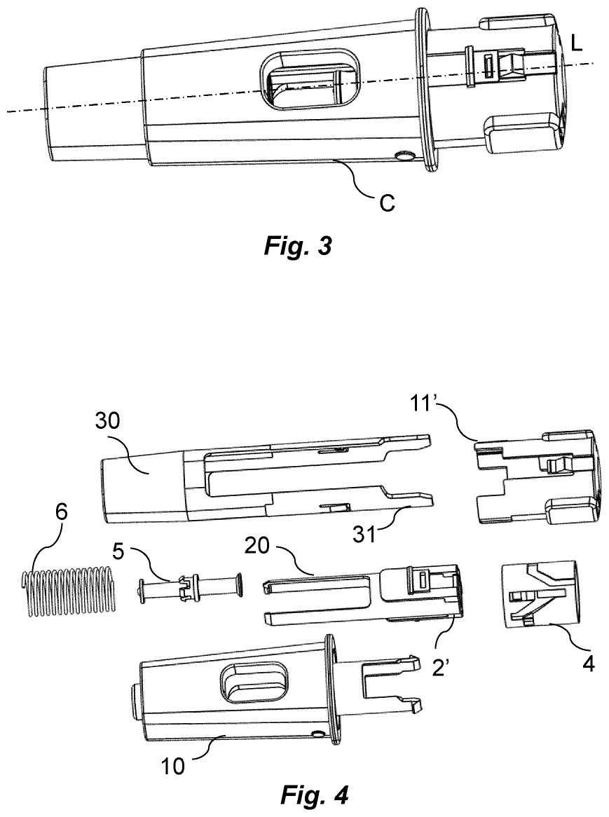

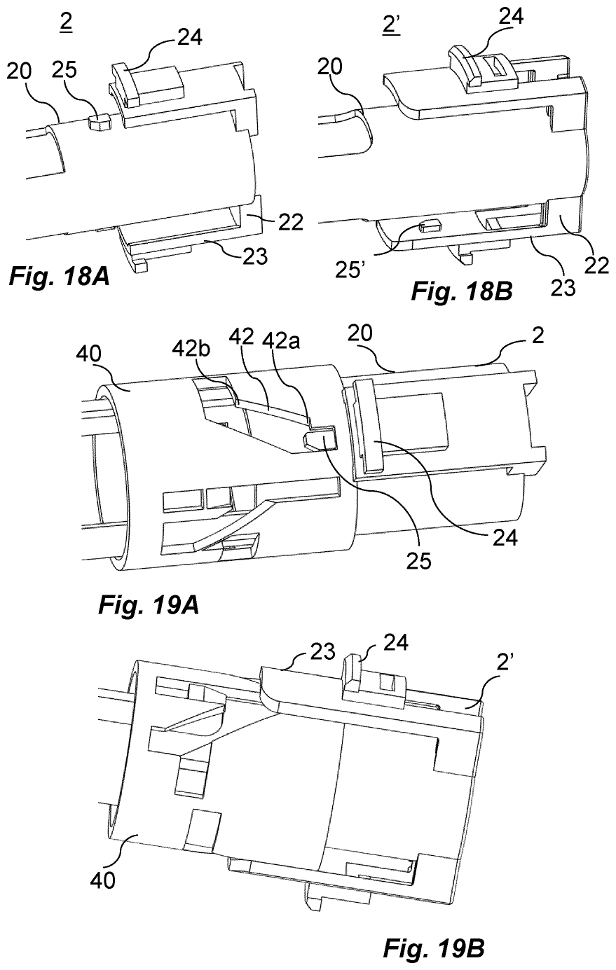

FIGS. 1-24 illustrate a sub-assembly of a medicament delivery device cassette unit. The sub-assembly of the invention provides a mechanism that a tubular carrier is moved in the proximal direction relative to the tubular body when the cassette unit is attached to the drive unit D (the cassette unit comprises the sub-assembly of the invention). Furthermore, the sub-assembly of the invention provides a safety mechanism that prevents an end user from getting injury from a medicament delivery member or prevents the medicament delivery member from being damaged/contaminated by the end user.

The term “medicament delivery device cassette unit” will sometimes be abbreviated as “cassette unit” in this description. It should be noted that both the term “medicament delivery device cassette unit” and the term “cassette unit” are used with the same meaning in this description.

The cassette unit is usually used with a medicament delivery device with two detachable parts, as shown in FIG. 22. There can be certain technical demands for making a medicament delivery device, such as a controllable medicament delivery speed; a high output force to deliver a high volume and/or high viscosity medicament; multiple indications for different medicament delivery stages; and/or data recording and/or communication, for example to track prescription adherence. However, the cost of manufacturing medicament delivery devices that can perform al functions and fulfil the technical demands described above is typically high, which makes the use of such medicament delivery device for frequent treatments less affordable. Medicament delivery devices with two detachable parts are usually made with a reusable part and a disposable part. The reusable part comprises most of the mechanical and/or electronic components. For example, the reusable part normally comprises a powerpack, namely, an assembly that comprises a power source and components that are configured to transfer the output force from the power source to a medicament. The disposable part is usually without electronics to lower the cost of manufacture. The disposable part, or is commonly referred to as a cassette unit c, is usually designed to accommodate a medicament container, e.g. a syringe or cartridge, that has a medicament contained within. The syringe or cartridge can be made of glass or plastic. The syringe or cartridge may comprise a rubber stopper sealing on a distal end of the syringe or cartridge, and a septum or medicament delivery member with a rubber sheath sealing a proximal end of the syringe or cartridge. Alternatively, the syringe or cartridge may be collapsible with only a septum or medicament delivery member (i.e. no stopper) with a rubber delivery member sheath sealing a proximal end of the syringe or cartridge.

The reusable part of the medicament delivery device, (i.e. a drive unit D), usually comprises a driver for expelling the medicament contained within the medicament container in the proximal direction of the medicament delivery device. The drive unit D may comprise a plunger rod for pushing on the medicament container. In one example, the stopper of the medicament container can therefore be moved in the proximal direction relative to the rest of the medicament container.

In another example, the plunger rod may collapse the medicament container by pushing the medicament container in the proximal direction relative to the cassette unit C. The drive unit D also comprises a power source, such as a spring, a gas canister, or a motor-driven gear set, for providing the power required to expel the medicament. The focus of the concept described herein is a sub-assembly for the medicament delivery device cassette, not the drive unit, and various types of drive units could be used with the cassette units with the sub-assembly described herein. As such, the drive unit will not be comprehensively described.

The sub-assembly of the invention comprises a tubular body 1; 1′; 1″. The sub-assembly further comprises one or more optional components, namely a tubular carrier 2; 2′; 2″, a lock member, a delivery member guard 3, a rotator 4; 4′, a delivery member assembly 5, and a biasing member 6, as shown in FIGS. 1-4. The optional components and their arrangement in the sub-assembly will be explained in detail later.

The tubular body 1; 1′; 1″ is configured to accommodate the medicament container in an outer shell 10. In one example, the tubular body is a single component. In another example, the tubular body 1; 1′; 1″ is formed by one or more components attached to each other. For example (see FIG. 2), the tubular body 1; 1′ comprises the outer shell 10 and a distal lid 11; 11′. In the former example, the medicament container can be inserted into the tubular body 1; 1′ from the proximal end of the tubular body 1; 1′; in the latter example, the medicament container can be inserted into the tubular body 1; 1′ from the distal end of the outer shell 10, then the distal lid 11; 11′ attaches to the distal end of the outer shell 10 to support the medicament container. In a preferred example, the distal lid 11; 11′ is immovably fixed to the distal end of the body 1; 1′ so that the distal lid 11; 11′ is immovable relative to the tubular body 1; 1′ in the direction of the longitudinal axis L and in a circumferential direction around the longitudinal axis L. For example, the distal lid 11; 11′ is attached to the distal end of the tubular body 1; 1′ by a snap-fit between a snap arm 10a on the tubular body 1; 1′ and a snap recess on the distal lid 11; 11′ (not shown), so that a movement of the medicament container in the distal direction relative to the tubular body 1; 1′ can be prevented.

If there is a requirement that the user needs to be able to see the medicament container, the outer shell 10 may comprise a window 12 aligned with the medicament container so that the end-user can observe the medicament through the window 12. Alternatively, the outer shell 10 may be transparent, so that the tubular body does not need the window 12. The cassette unit C is configured to be attached and detached to the drive unit D by a releasable connection between the tubular body 1; 1′; 1″ and the drive unit D when the end-user plans to carry out a medicament delivery operation. The tubular body 1; 1′; 1″ comprises a fastener configured to connect with a counter fastener on the drive unit D. In one example, the fastener and the counter fastener can form a bayonet connection, meaning that one of the tubular body 1; 1′; 1″ or the drive unit D has a bayonet protrusion with the other of the tubular body 1; 1′; 1″ and the drive unit D having a bayonet recess or cut-out. The bayonet recess or cut-out opens in the direction transverse to the longitudinal axis L. The bayonet recess or cut-out comprises a longitudinal section and a circumferential section. The longitudinal section extends in the direction of the longitudinal axis L and the circumferential section extends in the direction around the longitudinal axis L. The bayonet protrusion extends in the direction transverse to the longitudinal direction.

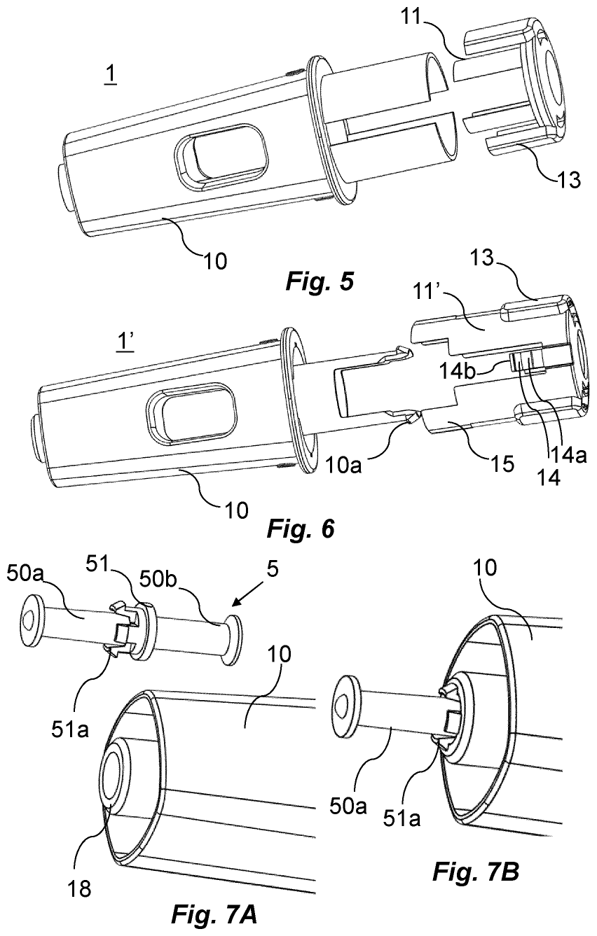

For example, the outer body 1; 1′ may have a bayonet connector 13 (in the example as shown in FIG. 22, the bayonet connector is the bayonet protrusion) as the fastener either on the outer shell of the tubular body or on the distal lid 11; 11′, such as the bayonet protrusions as shown in FIGS. 5-8 and FIG. 22; and the drive unit D would then have a counter bayonet connector DO (in the example as shown in FIG. 22, the counter bayonet connector is the bayonet recess). In this example, the tubular body 1; 1′; 1″ is configured to be axially inserted, along the longitudinal axis L, into a proximal part of the drive unit D, through the bayonet connector 13 and the counter bayonet connector DO; then the tubular body 1; 1′; 1″ needs to be rotated relative to the drive unit D, around the longitudinal axis L, so that the cassette unit C is property attached to the drive unit.

Alternatively, the connection between the tubular body 1; 1′; 1″ and the drive unit D can use another suitable connection, such as a snap-fit connection or thread connection; or the cassette unit may be completely received into the drive unit. In this example, the section of the drive unit for receiving the cassette unit may be a form-fit connection to receive the cassette unit. If the cassette unit is completely received into the drive unit, the shape of the body may be dependent on the shape of the section of the drive unit for receiving the cassette unit.

In one example where the tubular body 1; 1′ comprises the distal lid 11; 11′, the bayonet connector is arranged on the distal lid 11; 11′ and the tubular body 1; 1′ is configured to be attached to the drive unit D by a rotation around the longitudinal axis L relative to the drive unit D. The distal lid may have a strengthened wall 15 (as shown in FIG. 6), e.g. made from rigid or thick material, so that when the tubular body 1; 1′ is rotated relative to the drive unit D, the consequent torque transfer to the connector of the distal lid 11; 11′ will not damage the rest of the parts of the distal lid 11; 11′.

The shape of the distal lid 11; 11′ is typically dependent on the shape of the distal end of the tubular body 1; 1′. The distal lid 11; 11′ comprises a lid body that can be formed to follow the shape of the medicament container received within the tubular body 1; 1′, for example, a tubular lid body for use with a cylindrical medicament container. Further, the distal lid 11; 11′ typically comprises an opening axially aligned with a part of the distal end of the medicament container, so that when the cassette unit C is attached to the drive unit D, the plunger rod can pass through the opening and act on the medicament container for delivering the contained medicament.

The distal lid 11; 11′ optionally comprises a label, e.g. RFID tag, bar code, QR code, or mechanical code that contains information about the cassette unit C or the contained medicament, so that a reader in the drive unit D may get the information for further use.

The distal lid 11; 11′ optionally comprises a container support for supporting the medicament container received within the tubular body 1; 1′. The container support comprises a proximally directed surface. In one example, the container support is a flexible arm. The flexible arm can be formed by a cut-out on the lid body opening in the direction transverse to the longitudinal axis L; alternatively, the flexible arm may extend from the lid body in the distal direction. In this example, the container support can prevent an axial movement of the medicament container received within the tubular body 1; 1′ in the distal direction. The proximally directed surface of the container support can be formed on an inner surface of the flexible arm or can be formed on a distal tip of the flexible arm. The container support may also be used to tolerate variation in dimensions of the received medicament container, e.g. certain length and/or width of the received medicament container, regarding the engineering tolerance.

The sub-assembly of the invention provides the mechanism that the tubular carrier 2; 2′; 2″ is moved in the proximal direction relative to the tubular body when the cassette unit is attached to the drive unit D (the cassette unit comprises the sub-assembly of the invention).

The tubular carrier 2; 2′; 2″ is for holding a medicament container. The tubular carrier 2; 2′; 2″ is at least partially situated within the tubular body 1; 1′; 1″. The tubular carrier 2; 2′; 2″ and the tubular body 1; 1′; 1″ are coaxial. The tubular carrier 2; 2′; 2″ comprises a distally directed surface 24a for contacting with the drive unit D of the medicament delivery device when the tubular body 1; 1′; 1″ is attached to the drive unit D of the medicament delivery device. The tubular carrier 2; 2′; 2″ is movable relative to the tubular body 1; 1′; 1″ in the direction of the longitudinal axis L from a distal position to a proximal position by the drive unit D when the distally directed surface 24a is in contact with the drive unit D of the medicament delivery device.

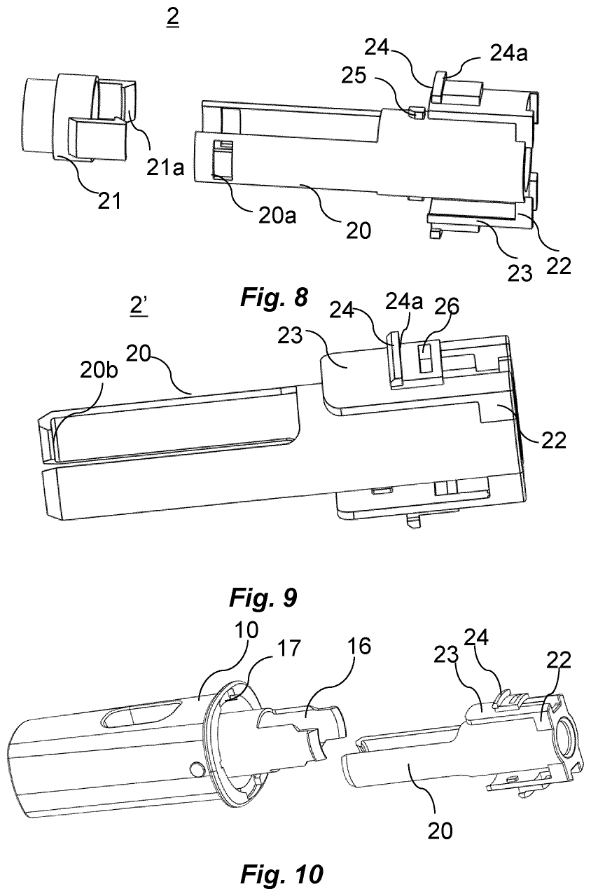

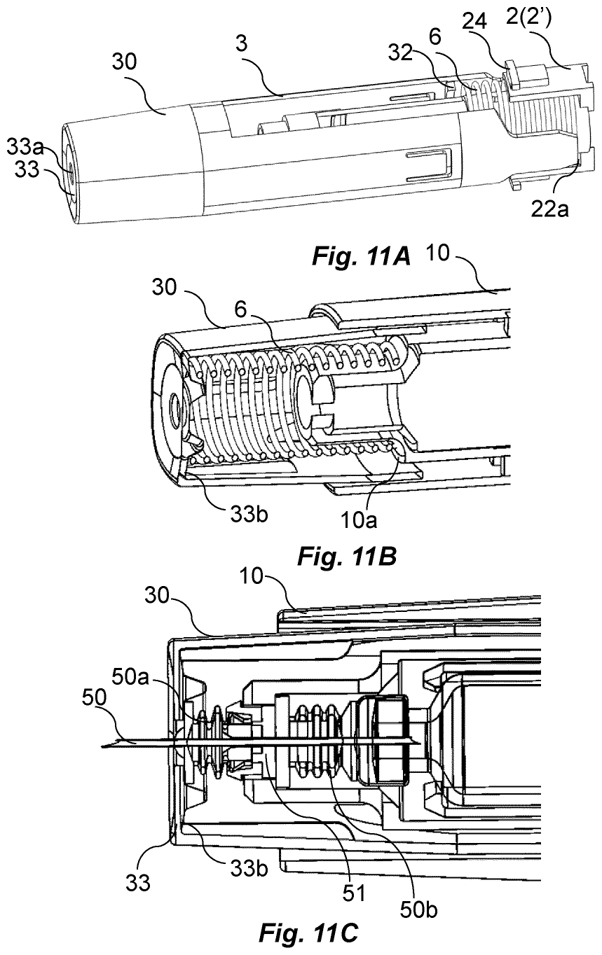

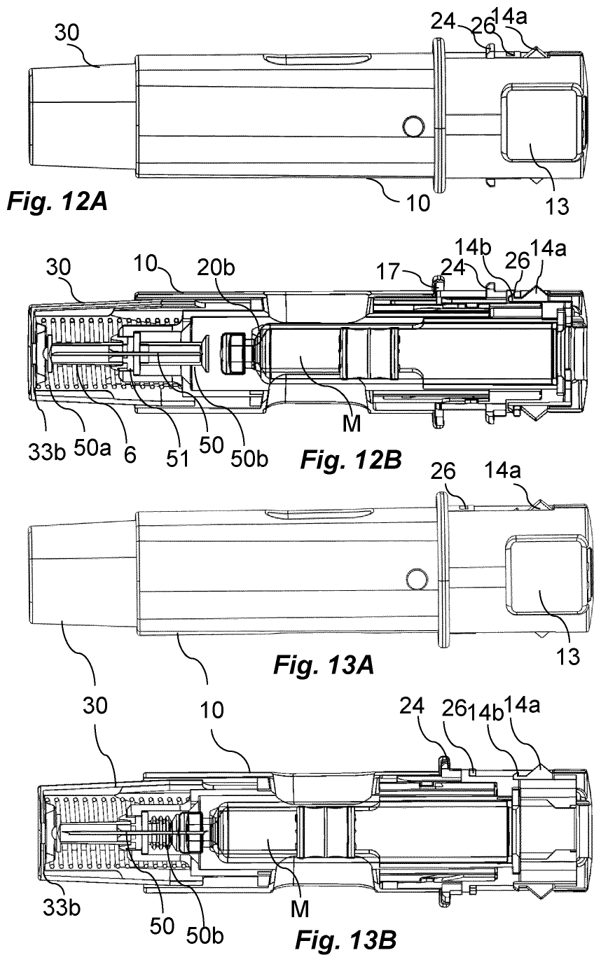

The tubular carrier 2; 2′; 2″ can be any suitable shape that can contain a medicament container. For example, the tubular carrier can be cylindrical, since many medicament containers, e.g. cartridge or syringes, have a cylindrical body. In another example, the tubular carrier is a ring or a circlip since the tubular carrier can simply hold on to a flange or a shoulder of the medicament container. Alternatively, as shown in FIGS. 8-9, the tubular carrier can be formed by two arms extending in the direction of the longitudinal axis L and a joint connected in between. In an example where the tubular carrier 2; 2′; 2″ comprises two arms, the tubular carrier 2; 2′; 2″ can be formed by a carrier body 20 comprising an arm section and a joint section. In this example, the tubular carrier 2 may comprise the carrier body 20 and a carrier cap 21, as shown in FIG. 8. The carrier body 20 is configured to receive a distal part of the medicament container and the carrier cap 21 is configured to receive a proximal part of the medicament container. The carrier cap 21 can be attached to the carrier body 20 through a snap-fit engagement, as shown in FIG. 8. In one example, the snap-fit engagement comprises a snap-fit arm 21a on the carrier cap 21 and a snap-fit recess or cut-out 20a on the carrier body 20. The snap fit engagement may also be accomplished with a snap-fit arm on the carrier body with a snap-fit recess or cut-out on the carrier cap. Alternatively, the carrier cap can be attached to the carrier body through a thread screw connection or a bayonet connection. The carrier cap can be any suitable shape, dependent on the shape of the carrier body and the shape of the medicament container, e.g. tubular or dome-shaped. An aperture is arranged at the proximal end of the carrier cap so that a delivery member can pass through. In a preferred example, the carrier cap comprises a support ledge extending from an inner surface in the direction transverse to the longitudinal axis L. The support ledge is configured to support the medicament container on the shoulder of the medicament container. In another example, the tubular carrier 2′ is formed simply by the carrier body 20 without the carrier cap. In this example, the carrier body 20 comprises a support finger 20b extending from an inner surface of the carrier body 20 at the proximal end of the carrier body 20, as shown in FIG. 9. The support finger 20b is configured to support the shoulder of the medicament container, as shown in FIG. 11C and FIG. 12B.

In a preferred example, the tubular carrier is engaged with the tubular body through a groove and ledge engagement. Preferably, the tubular carrier 2; 2′ comprises a ledge 22 extending from an outer surface of a wall of the tubular carrier 2; 2′, preferably, on an outer surface of a wall of the carrier body 20 in a radial direction relative to the longitudinal axis L. The groove for connecting the tubular carrier and the tubular body can be a recess arranged on an inner surface of the tubular body, or a cut-out (as shown in FIG. 10); alternatively, the groove can be arranged on the outer wall of the carrier body of the tubular carrier. In one example, A groove 16 is arranged on an inner wall of the tubular body 1; 1′, preferably on an inner wall of the outer shell 10 of the tubular body 1; 1′, as shown in FIG. 10. The ledge 22 is positioned within the groove when the tubular carrier 2; 2′ is attached to the tubular body 1; 1′. Alternatively, the tubular body comprises a ledge extending from an inner surface of a wall of the tubular body in a radial direction relative to the longitudinal axis L. A groove is arranged on the outer surface of the tubular carrier. The ledge is positioned within the groove. Furthermore, the groove is a recess or a slot. Alternatively, the tubular body comprises a ledge extending from the inner surface of a wall of the tubular body in a radial direction relative to the longitudinal axis. The tubular carrier comprises a counter ledge arranged on the outer surface of the tubular carrier. The ledge is positioned within the counter ledge. This engagement between the tubular carrier 2; 2′ and the tubular body 1; 1′; 1″ allows the tubular carrier 2; 2′ to be moved relative to the tubular body 1; 1′ in the direction of the longitudinal axis L without rotation.

In a preferred example, the tubular carrier 2; 2′; 2″ is configured to immovably hold the medicament container of the medicament delivery device, preferably in the direction of the longitudinal axis L, so that the medicament container is immovable relative to the tubular carrier 2; 2′; 2″ in the direction of the longitudinal axis L. Alternatively, instead of fixedly holding the medicament container, the tubular carrier only restricts the movement of the medicament container beyond a predetermined distance, namely, the medicament container of the medicament delivery device is movable relative to the tubular carrier for a predetermined distance, preferably, in the direction of the longitudinal axis L.

The tubular carrier 2; 2′; 2″ is configured to be moved in the proximal direction relative to the tubular body 1; 1′; 1″ by a part of the drive unit D, during the process of attaching the cassette unit comprising the sub-assembly of the invention to the drive unit D. The tubular carrier 2; 2′; 2″ comprises the distally directed surface 24a; 24a′. The distally directed surface 24a; 24a′ is configured to be in contact with a part of the drive unit D. Thus, the drive unit D can move the tubular carrier 2; 2′; 2″ in the proximal direction relative to the tubular body 1; 1′; 1″ during the process of attaching the cassette unit to the drive unit D (the cassette unit comprises the sub-assembly of the invention). In one example, the tubular carrier 2; 2′ comprises a flange 24 extending in the direction transverse to the longitudinal axis L. In one example, the flange extends from an outer surface of the carrier body. The distally directed surface 24a is defined by the flange 24, as shown in FIGS. 8-9. In a preferred example, the flange 24 extends out of the tubular body 1; 1′, namely, the flange 24 can be accessed from the outside of the tubular body 1; 1′. The flange can be arranged on an outer surface of carrier body 20, preferably, arranged closer to the distal end of the carrier body 20 than the proximal end of the carrier body 20. In another example, the tubular carrier 2; 2′ comprises an arm 23 extending in the direction of the longitudinal axis from the ledge 22 in the proximal direction relative to the carrier body 20. In this example, the flange 24 is arranged on an outer surface of the arm, as shown in FIGS. 8-9. In another example, as shown in FIG. 10, a recess 17 is arranged on the tubular body 1; 1′; 1″, the recess 17 is configured to receive the flange 24 when the tubular carrier 2; 2′ is in the proximal position.

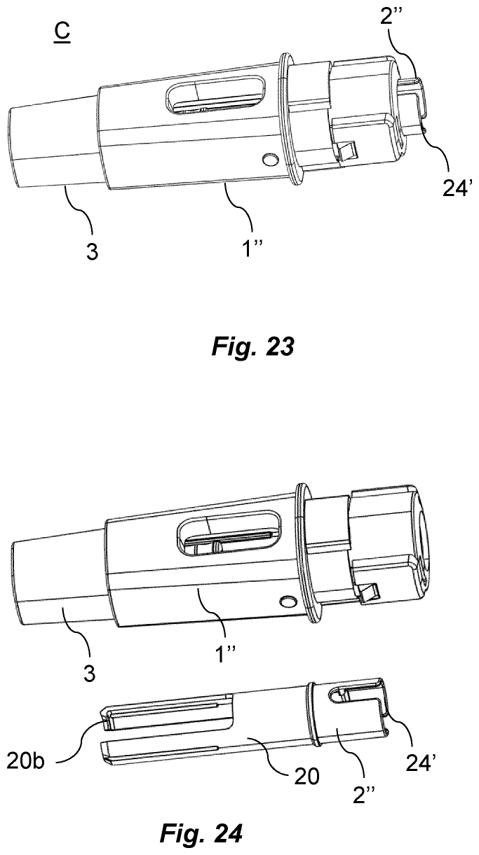

Alternatively, the distally directed surface is arranged at the distal end of the tubular carrier, e.g. an edge of the tubular carrier, as shown in FIGS. 23-24. In this example, the tubular carrier doesn't need to have the flange 24. In this example, the distally directed surface 24′ can be fully surrounded by the tubular body or extends from the distal end of the tubular body 1″, as shown in FIG. 23. In an example where the cassette unit that comprises the sub-assembly of the invention is attached to the drive unit by a bayonet connection and the tubular carrier 2; 2′; 2″ comprises the flange 24, the flange 24 is arranged next to the bayonet connector 13 (as the fastener of the tubular body); preferably, the flange 24 is circumferentially offset relative to the bayonet connector 13 of the tubular body in the direction of the longitudinal axis. In an example where the bayonet connector 13 is a bayonet protrusion, and the counter fastener of the driver unit D is a bayonet groove, when the bayonet connector 13 moves into a longitudinal part of the bayonet groove, the flange 24 abuts a proximal edge D1 of the drive unit D; once the bayonet connector 13 moves into a circumferential part of the bayonet groove, the flange 24 is pushed in the proximal direction relative to the drive unit by the proximal edge D1 of the drive unit D. Thus, the tubular carrier 2; 2′ is moved in the proximal direction relative to the tubular body 1; 1′ from the distal position to the proximal position.

Alternatively, the fastener and the counter fastener can be attached by a snap fit, a thread screw fit or a bayonet fit, any of mentioned arrangement is suitable for both the tubular carrier that comprises the flange 24, or the tubular carrier 2″ where the distally directed surface 24′ is arranged at the distal end of the tubular carrier 2″, as shown in FIGS. 23-24. In this example, the relative axial movement along the longitudinal axis between the fastener and the counter fastener enables a part of the drive unit D to push on the distally directed surface 24; 24′ in the proximal direction relative to the tubular body 1; 1′; 1″. Thus, the tubular carrier 2; 2′; 2″ is moved in the proximal direction relative to the tubular body 1; 1′; 1″ during the process of attaching the cassette unit C to the drive unit D (the cassette unit comprises the sub-assembly of the invention).

Furthermore, the sub-assembly of the invention may comprise an initial lock mechanism, so that the tubular carrier 2; 2′; 2″ is locked from moving to the proximal position until the cassette unit that comprises the sub-assembly is attached to the drive unit D. In one example, the initial lock arrangement comprises a flexible section 14 at the distal end of the tubular body 1′. The flexible section 14 can be arranged on the outer shell 10 or the distal lid 11′. In the example shown in FIG. 6, the flexible section 14 is arranged on the distal lid 11′. The flexible section 14 comprises a flexing part, a lock protrusion 14a, and a hook 14b. The flexing part can be a flexible arm or a part of the distal lid that is made of a resilient material. The lock protrusion 14a extends away from the tubular body 1′ in the radial direction relative to the longitudinal axis L. The hook 14b extends in a radial direction relative to the longitudinal axis L. A cut-out or recess 26 is arranged in the wall of the carrier body. Alternatively, the cut-out or recess 26 is arranged in a wall of arm 23 of the tubular carrier 2. The flexible section 14 is movable in the direction transverse to the longitudinal axis L relative to the tubular body 1′ between an engaged position where the hook 14b of the flexible section 14 is positioned within the cut-out or recess 26 of the tubular carrier 2′ and a released position where the hook 14b of the flexible section 14 is positioned out of the cut-out or recess 26 of the tubular carrier 2′. The flexible section 14 of the tubular body 1′ is moved from the engaged position to the released position when the lock protrusion 14a of the flexible section 14 is pressed, preferably from the outside of the tubular body by a part of the drive unit D, e.g. an inner wall of the drive unit D. Once the lock protrusion 14a is pressed, the deformation of the flexible section 14 causes the hook 14b to flex out from the cut-out or recess 26 of the tubular carrier 2′, so that the tubular carrier 2′ is not locked to the tubular body 1′. Thus, before the cassette unit that comprises the sub-assembly is attached to the drive unit D, the tubular carrier 2′ is locked to the tubular body 1′ by the engagement between the hook 14b of the flexible section 14 and the cut-out or recess 26 of the tubular carrier 2′; until the cassette unit that comprises the sub-assembly is attached to the drive unit D, the tubular carrier 2′ is released from the tubular body 1′, and is therefore able to move to the proximal position, as shown in FIG. 12B and FIG. 13B.

In another example, instead of the initial lock mechanism, the sub-assembly comprises a biasing member 6 facing to a proximally directed surface of the tubular carrier 2, and connecting to either a part of the tubular body 1 or a part of the delivery member guard 3. The biasing member 6 can be a spring or a flexible arm. In the former example, the biasing member 6 is adjacent to a proximally directed surface of the tubular carrier 2; 2′, and adjacent to either a part of the tubular body 1 or a part of the delivery member guard 3. In the latter example, the biasing member faces to the proximally directed surface of the tubular carrier, namely, the biasing member can be either adjacent to the proximally directed surface of the tubular carrier or spaced apart from the proximally directed surface of the tubular carrier, so that the biasing member will only block the tubular carrier when the tubular carrier moves in the proximal direction relative to the tubular body in a predetermined distance. Furthermore, in this example, the biasing member extends from either a part of the tubular body 1 or a part of the delivery member guard 3.

In one example, the proximally directed surface 22a of the tubular carrier 2 is defined by the ledge 22. The biasing member 6 is configured to support the tubular carrier 2 at the distal position, so that the tubular carrier 2 is prevented from unintentionally moving to the proximal position, e.g. by shaking or dropping. In a preferred example, the sub-assembly comprises the delivery member guard 3 coaxially attached to the tubular body 1; 1′; 1″ and being axially movable relative to the tubular body 1; 1′; 1″ along the longitudinal axis L. The delivery member guard 3 comprises a front shield 30 configured to surround a medicament delivery member of the medicament delivery device. The front shield 30 of the delivery member guard 3 is telescopic relative to the proximal end of the tubular body 1; 1′; 1″. In this example, a proximal end of the biasing member 6 is adjacent to a distally directed surface of the delivery member guard 3, and a distal end of the biasing member 6 is adjacent to the proximally directed surface of the tubular carrier 2, as shown in FIG. 11A. Thus, the biasing member 6 is configured to bias the delivery member guard 3 in the proximal direction of the tubular body 1 and configured to support the tubular carrier 2 at the distal position of the tubular carrier 2.

Alternatively, the biasing member 6 can only provide the function as a delivery member guard biasing member. In this example, the tubular body comprises a proximally directed surface adjacent to the distal end of the biasing member regardless the sub-assembly comprises the initial lock mechanism and/or the initial support or not.

In another example, the sub-assembly comprises the delivery member assembly 5. The delivery member assembly 5 comprises a delivery member 50 and a delivery member holder 51. The delivery member 50 is attached to the delivery member holder 51. The delivery member holder 51 is attached to the proximal end of the tubular body 1; 1′. This exemplified sub-assembly is suitable for the cassette unit with a cartridge as the medicament container.

In one example, as shown in FIGS. 7A-7B, the delivery member assembly 5 comprises a flexible sheath wrapping the delivery member. The flexible sheath is configured to seal at least one end of the delivery member. In a preferred example, the delivery member 50 is a needle. In this example, the needle 50 is held by the delivery member holder 51 and is wrapped by the flexible sheath 50a. The delivery member holder 51 comprises a fastener 51a, e.g. a hook, configured to engage with a proximal ledge 18 of the tubular body. Preferably, the proximal ledge defines a central passage for receiving the delivery member 50 and the flexible sheath. For example, the proximal ledge 18 radially extends relative to the longitudinal axis L and the proximal ledge 18 is a ring or two ledges facing one another.

In an example where the sub-assembly comprises the delivery member assembly 5, the proximal movement of the tubular carrier 2; 2′; 2″ can establish a fluid communication between the received medicament container and the delivery member 50. Alternatively in another example, the proximal movement of the tubular carrier 2; 2′; 2″ can move a proximal end of the medicament container out from the tubular body, so that a user can attach a medicament delivery device to the medicament container.

In one example, the tubular carrier 2; 2′; 2″ can establish a fluid communication between the received medicament container and the delivery member 50, as shown in FIGS. 12A-13B. In this example, the tubular carrier 2; 2′; 2″ is axially movable relative to the delivery member assembly 5 between the distal position and the proximal position. The tubular carrier 2; 2′; 2″ is configured to hold the medicament container M in a non-fluid connection with the delivery member 50 when the tubular carrier 2; 2′; 2″ is in the distal position; and the tubular carrier 2; 2′; 2″ is configured to hold the medicament container M in a fluid connection with the delivery member 50 when the tubular carrier 2; 2′; 2″ is in the proximal position.

When the cassette unit that comprises the sub-assembly is attached to the drive unit D, the drive unit D moves the tubular carrier 2; 2′; 2″ in the proximal direction relative to the tubular body 1; 1′; 1″, as mentioned above. In this example, preferably, the medicament container is fixed to the tubular carrier 2; 2′; 2″, so that when the tubular carrier 2; 2′; 2″ is moved in the proximal direction relative to the tubular body 1; 1′; 1″, the medicament container M also moves in the proximal direction relative to the tubular body 1; 1′; 1″. When the tubular carrier 2; 2′; 2″ reaches is proximal position, the delivery member 50 moves into the medicament container M so that a fluid communication between the delivery member 50 and the medicament container M is established. In one example, the flexible sheath 50b seals a distal end of the delivery member, so that the flexible sheath 50b is compressed between the delivery member holder 50 and a proximal end of the medicament container M when the fluid communication is established between the medicament container M and the delivery member, as shown in FIG. 13B. In one example, the delivery member assembly comprises a cap sealing a proximal end of the delivery member, so that the user can remove the cap before carrying out a medicament delivery operation. Alternatively, in another example, the delivery member assembly 5 comprises another flexible sheath 50a configured to seal the proximal end of the delivery member. In this example, either the proximal end of the delivery member guard 3 or the proximal end of the tubular body comprises a proximal wall with an opening sized to only allow the delivery member to pass. In one example the proximal end of the delivery member guard 3 comprises the proximal wall 33 that has an opening designed to allow the passage of the delivery member when the user carries out a medicament delivery operation by pushing the proximal wall 33 of the delivery member guard 3 on a medicament delivery site, the flexible sheath 50a that seals the proximal end of the delivery member is compressed between the delivery member holder 51 and a rear surface 33b of the proximal wall of the delivery member guard, as shown in FIG. 11C. Therefore, the delivery member 50 can extend out from the opening on the proximal wall 33 of the delivery member guard.

The sub-assembly of the invention can be operated with the following sequence of steps: providing a medicament delivery device cassette unit C; providing a drive unit D; and using the drive unit D to move the tubular carrier 2; 2′; 2″ of the medicament delivery device cassette unit C relative to the tubular body 1; 1′; 1″ of the medicament delivery device cassette unit C along the longitudinal axis L until the medicament delivery device cassette unit C is attached to the drive unit D.

Preferably, the cassette unit C is attached to the drive unit D by a bayonet connection or thread connection. The step by which the drive unit D moves the carrier of the medicament delivery device cassette unit C relative to the tubular body 1; 1′; 1″ of the medicament delivery device cassette unit C along the longitudinal axis until the medicament delivery device cassette unit C attaches to the drive unit D comprises the following: moving the medicament delivery device cassette unit C relative to the drive unit D along the longitudinal axis and rotating the medicament delivery device cassette unit C relative to the drive unit D around the longitudinal axis L.

Furthermore, the step of using the drive unit D to move the tubular carrier 2; 2′; 2″ comprises the steps of: connecting the tubular carrier 2; 2′; 2″ to a part of the drive unit D and moving the tubular carrier 2; 2′; 2″ during rotating the medicament delivery device cassette unit C relative to the drive unit D around the longitudinal axis L.

In an example where the sub-assembly comprises the delivery member assembly 5, the step of using the drive unit to move the tubular carrier 2; 2′; 2″ of the medicament delivery device cassette unit C relative to the tubular body 1; 1′; 1″ of the medicament delivery device cassette unit C along the longitudinal axis until the medicament delivery device cassette unit C attaches to the drive unit D comprising the step of using the movement of the tubular carrier 2; 2′; 2″ relative to the tubular body 1; 1′; 1″ to establish a fluid connection between a delivery member 50 to the medicament container M while the medicament delivery device cassette unit C attaches to the drive unit D.

The sub-assembly of the invention further provides the safety mechanism for a cassette unit. For providing the safety mechanism, the sub-assembly comprises the tubular body 1; 1, the delivery member guard 3, and the rotator 4; 4′. The safety mechanism is configured to block the delivery member guard 3 from moving in the distal direction relative to the tubular body 1; 1′; 1″ before the cassette unit that comprises the sub-assembly is attached to the drive unit D.

It should be noted that the safety mechanism can be used together with the mechanism that the tubular carrier 2; 2′; 2″ is configured to be moved in the proximal direction relative to the tubular body when the cassette unit is attached to the drive unit D (the cassette unit comprises the sub-assembly of the invention), as described above. Alternatively the safety mechanism can be used independently to the above-mentioned mechanism, and vice-versa.

The tubular body 1; 1′; 1″ and the delivery member guard 3 are similar to the above-mentioned tubular body; 1; 1′ and the delivery member guard 3. The delivery member guard 3 comprises the front shield 30 configured to protrude from the proximal end of the body 1; 1′. The front shield 30 is configured to shield a medicament delivery member integral with or being attachable to the medicament container when the medicament delivery device is used by the user. In a preferred example, the front shield 30 is a tubular-shaped element with an opening in the proximal end of the front shield 30, so that the front shield 30 of the delivery member guard 3 is coaxially attached to the tubular body 1; 1′; 1″ and is axially movable relative to the tubular body 1; 1′; 1″ along the longitudinal axis L between a distal position and a proximal position. Similarly, the front shield 30 of the delivery member guard is a similar shape to the tubular body 1; 1′; 1″ to make the device compact, but the delivery member guard can also be formed in any other suitable shape which is axially movable relative to the tubular body 1; 1′; 1″ along the longitudinal axis L.

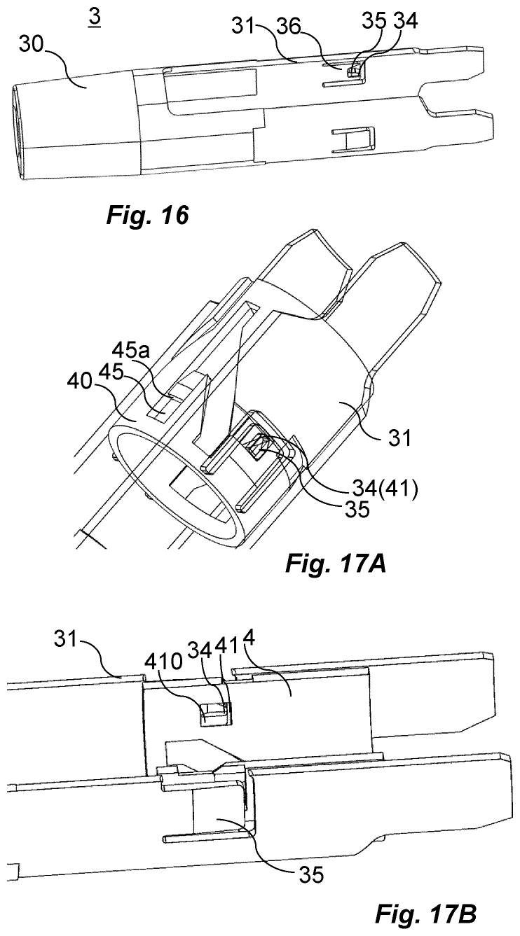

The delivery member guard 3 comprises an arm 31 extending from a distal end of the front shield 30 in the distal direction of the tubular body 1; 1′; 1″ of the sub-assembly. The delivery member guard 3 comprises a distally directed surface 34. Preferably, the delivery member guard 3 comprises a protrusion 35 defining the distally directed surface 34, as shown in FIG. 16, radially extending relative to the longitudinal axis L. In a preferred example, the protrusion 35 is arranged on a flexible section 36 of the delivery member guard.

In a preferred example, the delivery member guard 3 is partially arranged within the tubular body 1; 1′; 1″, namely the delivery member guard 3 is partially arranged within the outer shell 10 of the body 1; 1′. In this example, preferably, the arm 31 is fully arranged within the body 1; 1′; and the front shield 30 is fully or partially protruding from the proximal end of the tubular body 1; 1′; 1″ so that the front shield 30 is able to cover the medicament delivery member. In one example, the arm 31 of the delivery member guard 3 is configured to interact with a part of the drive unit D. For example, once the cassette unit C that comprises the sub-assembly has been assembled to the drive unit D, a further distal movement of the delivery member guard 3 may move the arm 31 into the drive unit D for releasing a plunger rod connected with a power source, e.g. a spring, a gas canister, a motor; or switching on the power source.

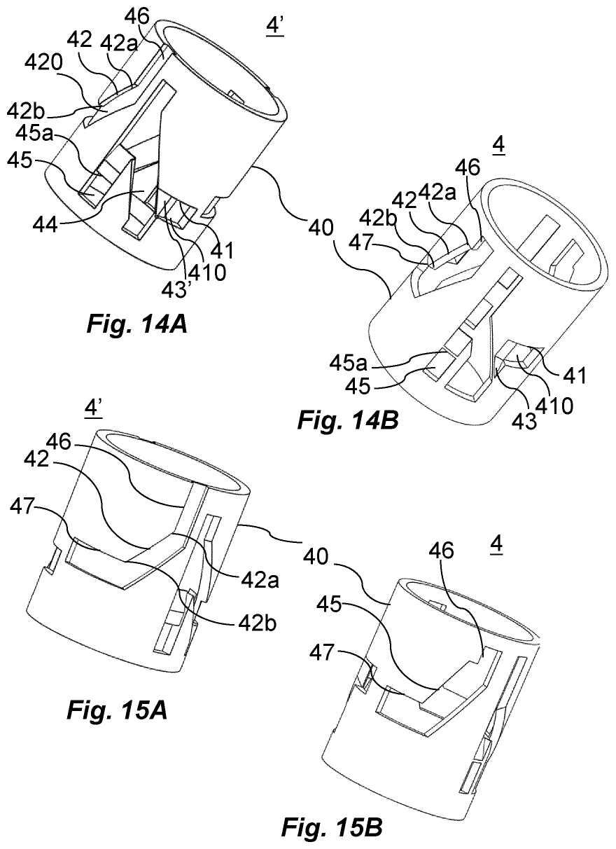

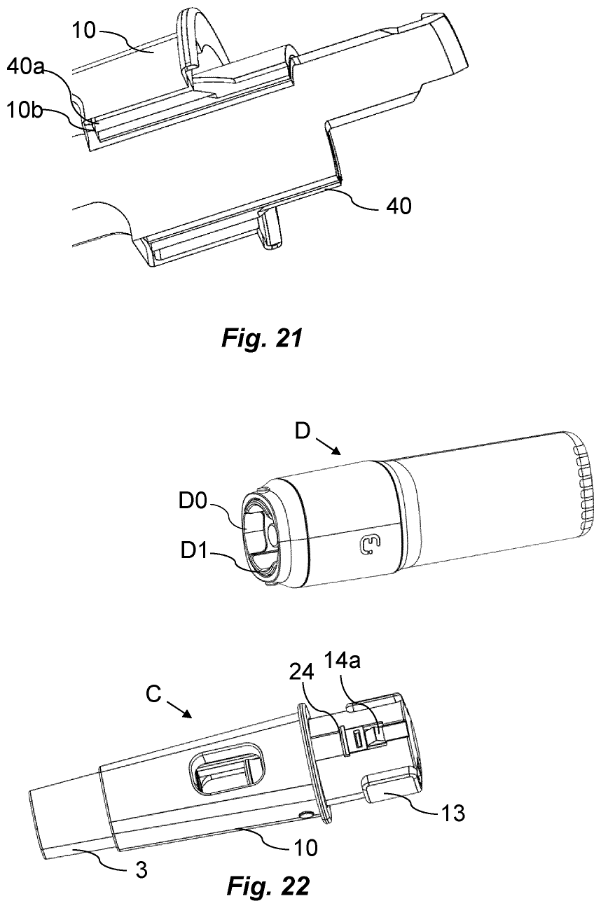

As shown in FIGS. 14A-15B, the rotator 4; 4′ is blocked from moving in any direction along the longitudinal axis L by the tubular body 1; 1′; 1″ tubular body 1; 1′; 1″. The rotator 4; 4′ comprises a proximally directed surface 41. The rotator 4; 4′ is configured to be rotated by the drive unit D of the medicament delivery device when the medicament delivery device cassette unit C is attached to the drive unit D, so that the rotator 4; 4′ is rotated relative to the delivery member guard 3 from a locked position where the distally directed surface 34 of the delivery member guard 3 is aligned to the proximally directed surface 41 of the rotator 4; 4′ to an unlocked position where the distally directed surface 34 of the delivery member guard 3 is not aligned from the proximally directed surface 41 of the rotator 4; 4′ so that the delivery member guard 3 is free to move in the distal direction relative to the rotator 4; 4′. The rotator 4; 4′ can be tubular-shaped, a ring or a circlip. In one example, the rotator 4; 4′ comprises a tubular rotator body 40. In one example, a proximal edge 40a of the tubular rotator body 40 is adjacent to an inner ledge 10b of the tubular body 1; 1′; 1″, as shown in FIGS. 17A-B and FIG. 21, and a distal edge of the rotator body is adjacent to a proximally directed surface of the distal lid 11; 11′, so that the rotator 4; 4′ is axially fixed to the tubular body 1; 1′; 1″. The proximally directed surface 41 of the rotator 4; 4′ can be arranged on a radially extending rib of the rotator or arranged on an edge of a cut-out or recess 410 of the rotator 4; 4′, as shown in FIGS. 14A-14B and FIG. 17B. The cut-out or recess 410 is arranged on the tubular rotator body 40.

In one example, the rotator comprises a ledge radially extending relative to the longitudinal axis L. The ledge is configured to engage with a part of the drive unit D so that when the drive unit D rotates relative to the tubular body of the sub-assembly, the rotator is rotated together with the drive unit relative to the tubular body of the sub-assembly.

In another example, the rotator 4; 4′ comprises a ledge 42 extending from a first end 42a to a second end 42b in a helical direction relative to the longitudinal axis L. In one example, the ledge is extending from an outer surface of the rotator body. Alternatively, the ledge 42 is defined by an edge of a cut-out or recess in a wall of the tubular rotator body 40. The ledge 42 is configured to engage with a part of the drive unit D, so that when the drive unit D of the medicament delivery device axially moves relative to the rotator 4; 4′ from a distal position where the part of the drive unit D is adjacent to the first end 42a of the ledge 42 and a proximal position where the part of the drive unit D is adjacent to the second end 42b of the ledge 42, thereby the rotator 4; 4′ is rotated.

Alternatively, in another example, the sub-assembly comprises a lock member. In one example, the lock member 2; 2′ can be integral to the tubular carrier 2; 2′; for example, the distal part of the tubular carrier 2; 2′ is arranged with the structure for acting as the lock member 2; 2′. In another example, the lock member can be an independent component. The latter example is suitable for the cassette unit that does not need a medicament container carrier, but the latter example can also be used in the cassette unit that comprises a medicament container carrier. The explanation of an example where the sub-assembly comprises the lock member set forth below will use the figures that the lock member is integral to the tubular carrier. However, since the tubular carrier, namely the component that is configured to receive a medicament container, is not necessary to form the safety mechanism, so that even the reference signs presented below will be the same as the reference signs, e.g. reference signs “2; 2′”, for the above-mentioned tubular carrier and the structures of the tubular carrier, the component and the structures below will be referred to the lock member instead of the tubular carrier. Furthermore, even the explanation set forth below will use the figures that the lock member is integral to the tubular carrier, those structures of the lock member are also suitable for forming the lock member that is a component independent to the tubular carrier.

Furthermore, some of the above-mentioned structural arrangements regarding tubular carrier 2; 2′ are applied to the lock member, either when the lock member is a part of the tubular carrier or is an independent component. Especially those structural arrangements that are not related to support the medicament container, such as the initial lock arrangement, and the connection between the biasing member 6 and the tubular carrier 2; 2′.

The lock member 2; 2′ is configured to directly engage with the rotator 4; 4′ and directly engage with a part of the drive unit D. Therefore, when the cassette unit attaches to the drive unit D, the drive unit D moves the lock member 2; 2′ and thus, rotates the rotator 4; 4′. The lock member 2; 2′ can be moved by the drive unit D in the above-mentioned way that moves the tubular carrier 2; 2′ relative to the tubular body 1; 1′; 1″ by the drive unit D. Therefore, only the way of rotating the rotator 4; 4′ by the lock member 2; 2′ will be explained in detail later on.

The lock member 2; 2′ is rotationally fixed to the tubular body 1; 1′; 1″, e.g. by the engagement between the ledge 22 of the lock member 2; 2′ and the groove 16 of the tubular body 1; 1′; 1″, as shown in FIG. 10.

The safety mechanism comprises a protrusion on either the rotator 4; 4′ or locking member 2; 2′ that radially extends relative to the longitudinal axis L and a ledge adjacent to the protrusion on the other component. Either the protrusion appears on the rotator 4; 4′ with the ledge on the locking member 2; 2′ or vice versa.

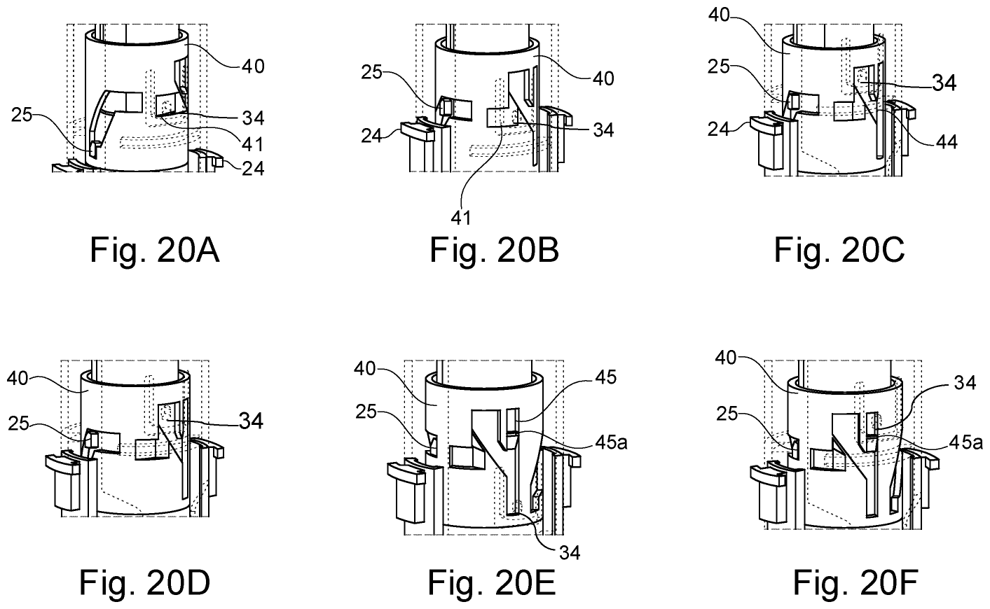

The ledge extending from a first end to a second end in a helical direction relative to the longitudinal axis L. In one example, the lock member 2; 2′ comprises the protrusion 25; 25′ as shown in FIG. 8 and FIG. 25. In an example where the lock member 2; 2′ is a part of the tubular carrier 2; 2′, the protrusion 25 can be arranged on the outer surface of the carrier body 20 (also named as lock member body 20); alternatively, the protrusion 25′ can be arranged on an inner surface of the arm 23 of the tubular carrier 2; 2′. In this example, the rotator 4; 4′ comprises the above-mentioned helically extending ledge 42. Instead of engaging with a part of the drive unit D, the ledge 42 of the rotator 4; 4′ in this example, is configured to engage with the protrusion 25; 25′ of the lock member 2; 2′. Therefore, when the lock member axially moves relative to the rotator 4; 4′ from the distal position where the protrusion 25; 25′ is adjacent to the first end 42a of the ledge 42, as shown in FIG. 19A and FIGS. 20-20B, and a proximal position where the protrusion 25; 25′ is adjacent to the second end 42b of the ledge 42 during attaching the cassette unit to the drive unit (the cassette unit comprises the sub-assembly of the invention), the axial movement of the lock member causes the rotator to rotate by the engagement between the protrusion 25; 25′ and the ledge 42. As shown in FIGS. 20A-20C, when the rotator 4; 4′ moves to the unlocked position, the distally directed surface 34 (shown by dash line in FIGS. 20A-20F) of the delivery member guard 3 is not aligned with the proximally directed surface 41 of the rotator 4; 4′, so that the delivery member guard 3 can move in the distal direction relative to the tubular body 1; 1′; 1″.

In an example where the rotator 4; 4′ comprises the helically extending ledge 42, regardless of whether the ledge 42 is configured to engage with a part of the drive unit D directly or engage with the protrusion 25; 25′ of the lock member 2; 2′, the first end 42a of the ledge optionally comprises a longitudinal part 48 extending in the direction of the longitudinal axis L, as shown in FIG. 14A and FIG. 15A. The longitudinal part 48 is configured to enable the rotation of the rotator 4; 4′ only when the cassette unit reaches a predetermined position relative to the drive unit D, e.g. the property attached position, so that if the user decides not to undertake the medicament operation before property attaches the cassette unit to the drive unit D, the rotator 4; 4′ stays in the locked position. Furthermore, optionally, a circumferential ledge 47 extends in a circumferential direction relative to the longitudinal axis L from the second end 41b of the ledge, as shown in FIG. 14A and FIG. 15A. When the rotator 4; 4′ is rotated by the drive unit D directly, the ledge 42 with the circumferential ledge 47 can replace the fastener 13 on the tubular body 1; 1′; 1″, namely, the cassette unit is attached to the drive unit D through the circumferential ledge 47 of the rotator 4; 4′ and the counter fastener of the drive unit D. When the rotator 4; 4′ is rotated by the lock member 2; 2, the circumferential ledge 47 is configured to block the lock member 2; 2′ from moving in the distal direction relative to the tubular body 1; 1′; 1″, as shown in FIGS. 20A-20F.

The sub-assembly optionally comprises a delivery member guard lockout mechanism configured to lock the delivery member guard in a proximal after-use position so that the delivery member guard cannot move in the distal direction relative to the tubular body 1; 1′; 1″. The front shield 30 of the delivery member guard 3 is configured to fully surround the delivery member 50 when the delivery member guard in the proximal after-use position. In this example, the rotator 4; 4′ comprises a track 44 arranged on the wall of the tubular rotator body 40. The track can be formed by a cut-out, or a recess or multiple transverse extending ledges arranged on the tubular rotator body. The protrusion 35 of the medicament delivery member guard 3 is configured to follow the track 44 of the rotator 4; 4′ when the medicament delivery member guard 3 moves in the distal direction relative to the tubular body 1; 1′; 1″ during the medicament delivery operation. The track 44 comprises a lockout section 45, and the protrusion 35 of the delivery member guard 3 is configured to move into the lockout section 45 as a result of the distal movement of the delivery member guard 3 towards the proximal after-use position of the delivery member guard 3, as shown in FIGS. 20D-20F. Thereafter, the distally directed surface 34 of the delivery member guard 3 is abutted by a blocking surface 45a of the rotator so that the delivery member guard 3 is prevented from moving in the distal direction relative to the rotator 4; 4′. In one example, the distal movement of the delivery member guard 3 is caused by the biasing member 6.

The rotator 4; 4′ optionally comprises a bridge surface 43; 43′ connects between the track 44 and the second end 42b of the ledge 42. The bridge surface is a ramp surface inclined from the track 44 to the second end 42b of the ledge 42. The bridge surface 43 can extend in the circumferential direction to the track 44, as shown in FIG. 14A; or extend in the longitudinal direction towards the track 44, as shown in FIG. 14B.

One example of initiating an operation of the medicament delivery device with the drive unit D and the cassette unit C, the delivery member guard 3 is moved by the user towards the drive unit D to a distal trigger position. This distal movement of the delivery member guard 3 reveals a medicament delivery member that connects with a contained medicament and may also trigger the drive unit D to output a force on the contained medicament. Therefore, when the delivery member guard 3 is in the distal trigger position, the medicament delivery member is revealed to the user. This distal movement of the delivery member guard 3 can be performed by pressing a proximal end of the front shield 30 of the delivery member guard 3 on a medicament delivery site.

In examples where the sub-assembly comprises the biasing member 6, the biasing member 6 is configured to bias the delivery member guard 3 in the proximal direction relative to the tubular body 1; 1′; 1″ of the sub-assembly. Once the medicament delivery operation has been performed, the user may remove the medicament delivery device from the medicament delivery site, and the biasing member 6 can therefore bias the delivery member guard 3 in the proximal direction of the tubular body 1; 1′; 1″ so that the front shield 30 of the delivery member guard 3 can shield the medicament delivery member.

The delivery member guard 3 is therefore biased to a proximal after-use position by the biasing member 6. The proximal after-use position can be the proximal position of the delivery member guard 3 as mentioned above or can be a second proximal position than the above mentioned the proximal position of the delivery member guard 3, dependent on the design of the medicament delivery device. For example, if the medicament delivery member of the medicament delivery device is configured to move beyond its initial position in the proximal direction of the medicament delivery device after use, like in a medicament delivery device comprising an auto needle insertion mechanism; the delivery member guard may also move into the second proximal position as a proximal after-use position.

The inventive concept has mainly been described above with reference to a few examples.

However, as is readily appreciated by a person skilled in the art, embodiments other than the ones disclosed above are equally possible within the scope of the inventive concept, as defined by the appended claims.

Some aspects of the invention are described in the clauses below.
1. A sub-assembly of a medicament delivery device cassette unit, the sub-assembly comprising:
   a tubular body for attaching to a drive unit of a medicament delivery device; wherein the tubular body extends along a longitudinal axis between a proximal end and a distal end; and
   a tubular carrier for holding a medicament container; wherein the tubular carrier is at least partially arranged within the tubular body; wherein the tubular carrier and the tubular body are coaxial; wherein the tubular carrier comprises a distally directed surface for contacting with the drive unit of the medicament delivery device when the tubular body being attached to the drive unit of the medicament delivery device; and wherein the tubular carrier is movable relative to the tubular body in the direction of the longitudinal axis from a distal positon to a proximal positon by the drive unit when the distally directed surface is in contact with the drive unit of the medicament delivery device.
2. The sub-assembly according to clause 1, wherein the tubular carrier is engaged with the tubular body through a groove and ledge engagement.
3. The sub-assembly according to clause 2, wherein the tubular carrier comprises a ledge extending from an outer surface of a wall of the tubular carrier in a radial direction relative to the longitudinal axis; wherein a groove is arranged on an inner wall of the tubular body; and wherein the ledge is positioned within the groove.
4. The sub-assembly according to clause 2, wherein the tubular body comprises a ledge extending from an inner surface of a wall of the tubular body in a radial direction relative to the longitudinal axis; wherein a groove is arranged on an outer surface of the tubular carrier; and wherein the ledge is positioned within the groove.
5. The sub-assembly according to clause 3 or 4, wherein the groove is a recess or a slot.
6. The sub-assembly according to clause 1, wherein the tubular body comprises a ledge extending from an inner surface of a wall of the tubular body in a radial direction relative to the longitudinal axis; wherein the tubular carrier comprises a counter ledge arranged on an outer surface of the tubular carrier; and wherein the ledge is positioned within the counter ledge.
7. The sub-assembly according to any one of the preceding clauses, wherein the tubular carrier is configured to immovably hold the medicament container of the medicament delivery device.
8. The sub-assembly according to any one of the preceding clauses, comprising a delivery member assembly; wherein the delivery member assembly comprises a delivery member and a delivery member holder; wherein the delivery member is attached to the delivery member holder; and wherein the delivery member holder is attached the proximal end of the tubular body.
9. The sub-assembly according to clause 8, wherein the tubular carrier is axially movable relative to the delivery member assembly between the distal position and the proximal position; wherein the tubular carrier is configured to hold the medicament container in a non-fluid connection with the delivery member when the tubular carrier is in the distal position; and wherein the tubular carrier is configured to hold the medicament container in a fluid connection with the delivery member when the tubular carrier is in the proximal position.
10. The sub-assembly according to any one of the preceding clauses, wherein the tubular body comprises a flexible section at the distal end of the tubular body; and wherein the flexible section comprises a hook extending in a radial direction relative to the longitudinal axis and a lock protrusion extending away from the tubular body in the radial direction relative to the longitudinal axis.
11. The sub-assembly according to clause 10, wherein the tubular carrier comprises a carrier body; wherein a cut-out or recess is arranged in a wall of the carrier body; and wherein the flexible section is movable in a direction transverse to the longitudinal axis relative to the tubular body between an engaged position where the hook of the flexible section is positioned within the cut-out or recess of the tubular carrier and a released position where the hook of the flexible section is positioned out of the cut-out or recess of the tubular carrier.
12. The sub-assembly according to clause 11, wherein the flexible section of the tubular body is moved from the engaged position to the released position when the lock protrusion of the flexible section is pressed from the outside of the tubular body.
13. The sub-assembly according to any one of the preceding clauses, wherein the sub-assembly comprises a delivery member guard telescopically arranged in the tubular body and a biasing member extending along the longitudinal axis between a proximal end and a distal end; and wherein the delivery member guard comprises a distally directed surface adjacent to the proximal end of the biasing member.
14. The sub-assembly according to clause 13, wherein the tubular carrier comprises a proximally directed surface adjacent to the distal end of the biasing member.
15. The sub-assembly according to clause 13, wherein the tubular body comprises a proximally directed surface adjacent to the distal end of the biasing member.
16. The sub-assembly according to any one of the preceding clauses, wherein the tubular carrier comprises a flange extending in a direction transverse to the longitudinal axis; wherein the distally directed surface is defined by the flange; and wherein the flange extends out of the tubular body.
17. The sub-assembly according to any one of the preceding clauses, wherein the tubular body comprises a fastener on an outer surface of the tubular body for attaching to a part of a medicament delivery device.
18. The sub-assembly according to clause 17, wherein the fastener is a bayonet connector.
19. The sub-assembly according to clause 17 or 18, when dependent on clause 16, wherein the flange of the tubular carrier is arranged next to the fastener.
20. The sub-assembly according to clause 19 when dependent clause 18, wherein the flange is circumferentially offset to the bayonet connector of the tubular body relative to the longitudinal axis.
21. The sub-assembly according to any one of the preceding clauses when dependent on clause 8, wherein the delivery member is a needle; and wherein the delivery member assembly comprises a flexible sheath wrapping the needle.
22. A medicament delivery device cassette unit comprising the sub-assembly according to any one of the preceding clauses.
23. A method of operating a medicament delivery device comprising a medicament delivery device cassette unit and a drive unit, wherein the medicament delivery device cassette unit comprises a body and a carrier; the method comprising the following sequence of steps of:
   providing a medicament delivery device cassette unit;
   providing a drive unit; and
   using the drive unit to move the carrier of the medicament delivery device cassette unit relative to the body of the medicament delivery device cassette unit along a longitudinal axis until the medicament delivery device cassette unit attaches to the drive unit.
24. The method according to clause 23, wherein the step using the drive unit to move the carrier of the medicament delivery device cassette unit relative to the body of the medicament delivery device cassette unit along the longitudinal axis until the medicament delivery device cassette unit attaches to the drive unit comprises the steps of: moving the medicament delivery device cassette unit relative to the drive unit in the direction of the longitudinal axis; and rotating the medicament delivery device cassette unit relative to the drive unit around the longitudinal axis.
25. The method according to clause 24, wherein the step of using the drive unit to move the carrier comprises the steps of connecting the carrier to a part of the drive unit; and moving the carrier during rotating the medicament delivery device cassette unit relative to the drive unit around the longitudinal axis.
26. The method according to any one of clauses 23-25, wherein the step of using the drive unit to move the carrier of the medicament delivery device cassette unit relative to the body of the medicament delivery device cassette unit along the longitudinal axis until the medicament delivery device cassette unit attaches to the drive unit comprising the step of using the movement of the carrier relative to the body to establish a fluid connection between a delivery member to the medicament container while the medicament delivery device cassette unit attaches to the drive unit.

Other aspects of the invention are described in the clauses below.
1. A sub-assembly of a medicament delivery device cassette unit, the sub-assembly comprising: a tubular body, a delivery member guard, and a rotator;
wherein the tubular body extends along a longitudinal axis between a proximal end and a distal end; wherein the tubular body is tubular;
wherein the delivery member guard is telescopically arranged within the tubular body; wherein the delivery member guard comprises a distally directed surface;
wherein the rotator is blocked from moving in either direction along the longitudinal axis by the tubular body; and wherein the rotator comprises a proximally directed surface; and wherein the rotator is configured to be rotated by a drive unit of the medicament delivery device when the medicament delivery device cassette unit is attached to the drive unit, so that the rotator is rotated relative to the delivery member guard from a locked position where the distally directed surface of the delivery member guard is aligned to the proximally directed surface of the rotator to a unlocked position where the distally directed surface of the delivery member guard is not aligned from the proximally directed surface of the rotator so that the delivery member guard is free to move in the distal direction relative to the rotator.
2. The sub-assembly according to clause 1, wherein the rotator comprises a tubular rotator body.
3. The sub-assembly according to clause 1 or 2, wherein the rotator comprises a ledge radially extending relative to the longitudinal axis; and wherein the ledge is configured to engage with a part of the drive unit, so that when the drive unit rotates relative to the tubular body of the sub-assembly, the rotator is rotated together with the drive unit relative to the tubular body of the sub-assembly.
4. The sub-assembly according to clause 1 or 2, wherein the rotator comprises a ledge extending from a first end to a second end in a helical direction relative to the longitudinal axis; and wherein the ledge is configured to engage with a part of the drive unit, so that when the drive unit of the medicament delivery device axially moves relative to the rotator from a distal position where the part of the drive unit is adjacent to the first end of the ledge and a proximal position where the part of the drive unit is adjacent to the second end of the ledge, thereby the rotator is rotated.
5. The sub-assembly according to clause 1 or 2, comprising a lock member; wherein the lock member is rotationally fixed to the tubular body; wherein one of the rotator and the lock member comprises a protrusion radially extending relative to the longitudinal axis; wherein the other one of the rotator and the lock member comprises a ledge adjacent to the protrusion; wherein the ledge extending from a first end to a second end in a helical direction relative to the longitudinal axis; wherein the lock member is axially movable relative to the rotator from a distal position where the protrusion is adjacent to the first end of the ledge and a proximal position where the protrusion is adjacent to the second end of the ledge; and wherein the lock member is configured to engage with a part of the drive unit, so that when the drive unit of the medicament delivery device axially moves relative to the tubular body, the lock member moves together with the drive unit, thereby the rotator is rotated by the engagement between the protrusion and the ledge.
6. The sub-assembly according to any one of the preceding clauses, wherein one of the rotator and the delivery member guard comprises a protrusion, and the other one of the rotator and the delivery member guard comprises a cut-out or recess; and wherein the distally directed surface of the delivery member guard and the proximally directed surface of the rotator are defined by the protrusion and the cut-out or recess respectively.
7. The sub-assembly according to clause 6 when dependent on clause 2, or clause 6 when dependent on clause 2 and dependent on any one of clauses 3-5, wherein the cut-out or recess is arranged on the tubular body of the rotator.
8. The sub-assembly according to any one of clause 3-5 when dependent on clause 2 or according to any one of clause 3-5 when dependent on clause 2 and dependent on clause 6 or 7, wherein the ledge is extending from the tubular body of the rotator in the radial direction relative to the longitudinal axis.
9. The sub-assembly according to any one of clause 3-5 when dependent on clause 2 or according to any one of clause 3-5 when dependent on clause 2 and dependent on clause 6 or 7, wherein a second cut-out or recess is arranged on the tubular body of the rotator; and wherein the ledge is defined by an edge of the second cut-out or recess.
10. The sub-assembly according to any one of clauses 3-5 or 8-9, or according to any one of clauses 6 or 7 when dependent on any one of clauses 3-5, wherein a circumferential ledge extends in a circumferential direction relative to the longitudinal axis from the second end of the ledge.
11. The sub-assembly according to any one of clauses 3-5 or 8-10, or according to any one of clauses 6 or 7 when dependent on any one of clauses 3-5, wherein the first end of the ledge comprises a longitudinal part extending in the direction of the longitudinal axis.
12. The sub-assembly according to clause 5 or according to any one of clauses 6-11 when dependent on clause 5, comprising a biasing member extending along the longitudinal axis between a proximal end and a distal end; wherein the delivery member guard comprises a distally directed surface; wherein the lock member comprises a proximally directed surface; and wherein the proximal end of the biasing member is adjacent to the distally directed surface of the delivery member guard; and wherein the distal end of the biasing member is adjacent to the proximally directed of the lock member.
13. The sub-assembly according to any one of the preceding clauses, wherein the tubular body comprises a fastener on an outer surface of the tubular body for attaching to a part of a medicament delivery device.
14. The sub-assembly according to clause 13, wherein the fastener is a bayonet connector.
15. The sub-assembly according to clause 13 or 14, when dependent on clause 5 or according to clause 13 or 14, when dependent on any one of clauses 6-12 when dependent on clause 5, wherein the lock member comprises a lock member body and a flange extending from the lock member body; wherein the flange protrudes from the outer surface of the tubular body; and wherein the flange is arranged next to the fastener.
16. The sub-assembly according to clause 15 when dependent clause 14, wherein the flange is circumferentially offset to the bayonet connection of the tubular body relative to the longitudinal axis.
17. The sub-assembly according to any one of the preceding clauses, wherein the tubular body comprises a flexible arm extending from the distal end of the tubular body towards the proximal end of the tubular body; and wherein the flexible arm comprises a hook extending in the radial direction relative to the longitudinal axis, and a lock protrusion extending to the outside of the tubular body in the radial direction relative to the longitudinal axis.
18. The sub-assembly according to clause 17, when dependent on clause 5 or according to clause 17 when dependent on any one of clauses 6-12 when dependent on clause 5, wherein the lock member comprises a lock member body; wherein a cut-out or recess is arranged in a wall of the lock member body; and wherein the hook of the flexible section of the tubular body is configured to position within the cut-out or recess; and wherein the lock protrusion of the flexible section is configured to engage with a part of a driver unit of the medicament delivery device, so that when the tubular body is attached to the drive unit of the medicament delivery device, a section of the drive unit presses the lock protrusion inwardly relative to the tubular body, thus the hook flexes out from the cut-out or recess.
19. A medicament delivery device cassette unit comprising the sub-assembly according to any one of the preceding clauses.
20. A method of operating a medicament delivery device comprising a medicament delivery device cassette unit and a drive unit; wherein the delivery device cassette unit comprises a tubular body, a delivery member guard, and a rotator; the method comprising the following sequence of the steps of:
   providing the medicament delivery device cassette unit;
   providing the drive unit; and using the drive unit to rotate the rotator of the medicament delivery device cassette unit relative to the tubular body of the medicament delivery device cassette unit whilst attaching the medicament delivery device cassette unit to the drive unit.
21. The method according to clause 20, wherein the step using the drive unit to rotate the rotator of the medicament delivery device cassette unit relative to the tubular body of the medicament delivery device cassette unit whilst attaching the medicament delivery device cassette unit to the drive unit comprises the following sequence of the steps of: moving the medicament delivery device cassette unit relative to the drive unit along a longitudinal axis; and rotating the medicament delivery device cassette unit relative to the drive unit around the longitudinal axis.
22. The method according to clause 21, wherein the step of using the drive unit to rotate the rotator of the medicament delivery device cassette unit relative to the tubular body of the medicament delivery device cassette unit whilst attaching the medicament delivery device cassette unit to the drive unit comprises the steps of: establishing a connection between the rotator and a part of the drive unit; and rotating the medicament delivery device cassette unit relative to the drive unit around the longitudinal axis, whereby the rotator is rotated by the drive unit through the connection between the rotator and the drive unit.
23. The method according to clause 22, wherein the step of establishing a connection between the rotator and a part of the drive unit comprises the steps of: indirectly connecting the rotator to the part of the drive unit via a lock member of the medicament delivery device cassette unit; and rotating the medicament delivery device cassette unit relative to the drive unit around the longitudinal axis, thereby the lock member is axially moved in the proximal direction of the rotator by the drive unit, and thereby the rotator is rotated by the lock member.

1-14. (canceled)

15. A sub-assembly of a medicament delivery device cassette unit, the sub-assembly comprising:
a tubular body for attaching to a drive unit of a medicament delivery device, wherein the tubular body extends along a longitudinal axis between a proximal end and a distal end; and
a tubular carrier for holding a medicament container, wherein the tubular carrier is at least partially arranged within the tubular body, wherein the tubular carrier and the tubular body are coaxial, wherein the tubular carrier comprises a distally directed surface for contacting with the drive unit of the medicament delivery device when the tubular body being attached to the drive unit of the medicament delivery device,
wherein the tubular body comprises a flexible section at the distal end of the tubular body, and wherein the flexible section comprises a hook extending in a radial direction relative to the longitudinal axis and a lock protrusion extending away from the tubular body in the radial direction relative to the longitudinal axis,
wherein the tubular carrier comprises a carrier body, wherein a cut-out or recess is arranged in a wall of the carrier body, and wherein the flexible section is movable in a direction transverse to the longitudinal axis relative to the tubular body between an engaged position where the hook of the flexible section is positioned within the cut-out or recess of the tubular carrier and a released position where the hook of the flexible section is positioned out of the cut-out or recess of the tubular carrier, and
wherein the flexible section of the tubular body is moved from the engaged position to the released position when the lock protrusion of the flexible section is pressed from the outside of the tubular body.

16. The sub-assembly according to claim 15, wherein the tubular carrier is engaged with the tubular body through a groove and ledge engagement.

17. The sub-assembly according to claim 16, wherein the tubular carrier comprises a ledge extending from an outer surface of a wall of the tubular carrier in a radial direction relative to the longitudinal axis, wherein a groove is arranged on an inner wall of the tubular body, and wherein the ledge is positioned within the groove.

18. The sub-assembly according to claim 16, wherein the tubular carrier is configured to immovably hold the medicament container of the medicament delivery device.

19. The sub-assembly according to claim 16, comprising a delivery member assembly, wherein the delivery member assembly comprises a delivery member and a delivery member holder, wherein the delivery member is attached to the delivery member holder, and wherein the delivery member holder is attached the proximal end of the tubular body.

20. The sub-assembly according to claim 19, wherein the tubular carrier is axially movable relative to the delivery member assembly between the distal position and the proximal position, wherein the tubular carrier is configured to hold the medicament container in a non-fluid connection with the delivery member when the tubular carrier is in the distal position, and wherein the tubular carrier is configured to hold the medicament container in a fluid connection with the delivery member when the tubular carrier is in the proximal position.

21. The sub-assembly according to claim 16, wherein the sub-assembly comprises a delivery member guard telescopically arranged in the tubular body and a biasing member extending along the longitudinal axis between a proximal end and a distal end, and wherein the delivery member guard comprises a distally directed surface adjacent to the proximal end of the biasing member.

22. The sub-assembly according to claim 21, wherein the tubular body comprises a proximally directed surface adjacent to the distal end of the biasing member.

23. The sub-assembly according to claim 16, wherein the tubular carrier comprises a flange extending in a direction transverse to the longitudinal axis, and wherein the distally directed surface is defined by the flange, and wherein the flange extends out of the tubular body.

24. The sub-assembly according to claim 16, wherein the tubular body comprises a fastener on an outer surface of the tubular body for attaching to a part of a medicament delivery device.

25. The sub-assembly according to claim 24, wherein the fastener is a bayonet connector.

26. The sub-assembly according to claim 24, wherein the flange of the tubular carrier is arranged next to the fastener.

27. The sub-assembly according to claim 16, wherein the tubular carrier is movable relative to the tubular body in the direction of the longitudinal axis from a distal position to a proximal position by the drive unit when the distally directed surface is in contact with the drive unit of the medicament delivery device.

28. The sub-assembly according to claim 27, the distally directed surface is in contact with the drive unit of the medicament delivery device when the tubular body is attached to the part of the medicament delivery device via the fastener.
